(19) United States
(12) Patent Application Publication
(10) Pub. No.: US 2011/0311492 A1
(43) Pub. Date: Dec. 22, 2011

(54) MAINTENANCE/EXPANSION OF HSC's

(76) Inventors: Catherine Verfaillie, Leuven (BE); Shannon Mychel Buckley, New York, NY (US); Satish Khurana, Haryana (IN)

(21) Appl. No.: 13/132,283

(22) PCT Filed: Dec. 1, 2009
(86) PCT No.: PCT/US2009/066271

§ 371 Date: Jul. 21, 2011

Related U.S. Application Data
(60) Provisional application No. 61/118,819, filed on Dec. 1, 2008.

(51) Int. Cl. A61K 35/12; A61P 7/00; A61K 38/18; C12N 5/0789; A61K 38/02

(52) U.S. Cl. 424/93.7; 435/375; 514/13.5; 514/7.6

(57) ABSTRACT

The invention is related to methods for culturing stem cells, more particularly hematopoietic stem cells (HSC). The invention relates to methods for HSC maintenance and/or expansion through the use of soluble factors. The invention is also directed to cells produced by the methods of the invention. The cells are useful, among other things, for treatment of disorders or diseases (e.g. leukemia). The invention also relates to the development of small molecules that may increase HSC self renewal in vitro and in vivo.

### RELATED APPLICATION

This application claims priority from U.S. Provisional Application Ser. No. 61/118,819 filed Dec. 1, 2008, which application is herein incorporated by reference.

### GOVERNMENT FUNDING

The invention described herein was developed with support from the National Institutes of Health grant number PO1-CA-65493-012. The U.S. Government has certain rights in the invention.

### FIELD OF THE INVENTION

The invention is related to methods for culturing somatic stem cells, more particularly hematopoietic stem cells (HSC). The invention relates to methods for HSC maintenance and/or expansion and the use of one or more soluble factors (with or without the addition of one or more cytokines and/or growth factors) to increase the retention/maintenance and/or expansion of HSC/KLS cells in vitro. The invention is also directed to cells produced by the methods of the invention. The cells are useful, among other things, for treatment of disorders or diseases (e.g. leukemia). The invention also relates to the development of small molecules that may increase HSC self-renewal in vitro and in vivo.

### BACKGROUND OF THE INVENTION

Hematopoiesis is the process by which hematopoietic stem cells give rise to all hematopoietic lineages during the lifetime of an individual. To sustain life-long hematopoiesis, HSC must self-renew to maintain or expand the HSC pool, and they must differentiate to form committed hematopoietic progenitor cells (HPCs) that progressively lose self-renewal potential and become increasingly restricted in their lineage potential. A combination of extrinsic and intrinsic signals are thought to converge to regulate HSC differentiation versus self-renewal decisions, but the molecular mechanisms that regulate these processes are poorly understood.

A multitude of cytokines have been cloned that affect HSCs and HPCs; however, to date none of these, alone or in combination, can induce the symmetrical, self-renewing HSC division in vitro that is needed for HSC expansion. Recently, several novel regulators of HSC fate decisions have been identified. For instance, overexpression of HoxB4 results in expansion of murine and human HSCs with an increased competitive repopulation potential; novel extrinsic regulators implicated in self-renewal of HSCs include Notch, Wnt, and the morphogens, sonic hedgehog (Shh) and bone morphogenetic protein (BMP)-4. While the discovery of these novel regulators provides credence to the hypothesis that extrinsic and intrinsic signals can influence HSC fate, a more global gene and/or protein expression analysis of human HSC provided additional preliminary insight into pathways that support HSC self-renewal (Eckfeldt et al., P1oS, 2005, 3; 8:1449-1458). Since several diseases/disorders need a large amount of HSCs for transplantation and this HSC-availability is insufficient, there is an increased need for new methods to expand HSCs. Such methods would greatly facilitate the development of new therapies for hematopoietic disorders like leukemia for which currently no sufficient therapy exists. New methods for HSC-expansion would potentially overcome the problems with Graft Versus Host Disease (GVHD) complications correlated with the limited number of HSC currently available for transplantation, by making it possible to choose the best compatible donor, regardless of the amount of cells available (Schoemans H et al., Bone Marrow transplantation, 2006, 38(2): 83-93).

### SUMMARY OF THE INVENTION

One embodiment provides a method to maintain/expand hematopoietic stem cells (HSCs) comprising contacting HSCs with at least one or more exogenous factors discussed herein in particular those selected from TFPI, DEFCR3, SERPINE2, COL18A1, BGLAP, COL8A1, INHBA, LTBP2, MAC30, a biologically active fragment or derivative thereof so as to maintain/expand said HSCs. In one embodiment, the derivative is at least about 80%, about 85%, about 90%, about 95% or about 100% identical in sequence. In one embodiment, the factor is TFPI. In another embodiment, the factor is SERPINE2 and/or Tfpi. The exogenous factors can be used alone or in any combination of exogenous factors. In one embodiment, the HSCs are umbilical cord or bone marrow HSCs.

In another embodiment, the HSCs are further contacted with one or more growth factors or cytokines, including, but not limited to, Tpo and/or SCF and/or Ftt3L, IL6+IL6-receptor, and/or IL3 (e.g., low dose).

In one embodiment, the maintained/expanded HSCs are long-term-repopulating (LTR-) HSCs, including competitive repopulation (CR)-long-term-repopulating (LTR-) HSC. In another embodiment, the maintained/expanded HSCs are short-term-repopulating (STR-) HSCs.

In another embodiment, the contacting is carried out in vitro. In one embodiment, the HSCs are of human origin.

Another embodiment provides cells produced according to the methods described herein and compositions comprising those cells as well as compositions comprising HSCs and one or more factors selected from TFPI, DEFCR3, SERPINE2, COL18A1, BGLAP, COL8A1, INHBA, LTBP2, MAC30, a biologically active fragment or derivative thereof. In one embodiment, the factor is TFPI. In another embodiment, the factor is SERPINE2 and/or Tfpi. Another embodiment provides a composition further comprising a cytokine or growth factor and/or cell culture medium or a pharmaceutically acceptable carrier.

One embodiment provides a method to prepare a composition comprising combining one or more factors selected from TFPI, DEFCR3, SERPINE2, COL18A1, BGLAP, COL8A1, INHBA, LTBP2, MAC30, a biologically active fragment or derivative thereof with HSCs. In one embodiment, the factor is TFPI. In another embodiment, the factor is SERPINE2 and/or Tfpi. One embodiment provides for the addition of a cytokine or growth factor and/or cell culture medium or a pharmaceutically acceptable carrier.

One embodiment provides a method to treat a non-malignant blood disorder, a metabolic storage disorder or cancer comprising administering to a subject in need thereof HSCs which have been contacted with one or more factors selected from TFPI, DEFCR3, SERPINE2, COL18A1, BGLAP, COL8A1, INHBA, LTBP2, MAC30, a biologically active fragment or derivative thereof so as to treat a non-malignant blood disorder, a metabolic storage disorder or cancer in the subject. In one embodiment, the factor is TFPI. In another embodiment, the factor is SERPINE2 and/or Tfpi. In one embodiment, the HSCs have further been contacted with a cytokine including SCF and/or Tpo.

In one embodiment, the non-malignant blood disorder is selected from the group of immunodeficiencies comprising SCID, fanconi's anemia, aplastic anemia, or congenital hemoglobinopathy.

In another embodiment, the metabolic storage disease is Hurler's disease, Hunter's disease, or mannosidosis.

In one embodiment, the cancer is selected from the group of hematological malignancies comprising acute leukemia, chronic leukemia, lymphoma, multiple myeloma, myelodysplastic syndrome, or non-hematological cancer. In one embodiment, the chronic leukemia is myeloid or lymphoid. In another embodiment, the lymphoma is Hodgkin's or non-Hodgkin's lymphoma. In another embodiment, the non-hematological cancer is breast carcinoma, colon carcinoma, neuroblastoma, or renal cell carcinoma.

In one embodiment, the subject has been treated with chemotherapy or radiation. In another embodiment, the subject has lost HSCs.

One embodiment provides a method to treat a non-malignant blood disorder, a metabolic storage disorder or cancer comprising administering to a subject in need thereof one or more factors selected from TFPI, DEFCR3, SERPINE2, COL18A1, BGLAP, COL8A1, INHBA, LTBP2, MAC30, a biologically active fragment or derivative thereof so as to treat a non-malignant blood disorder, a metabolic storage disorder or cancer in the subject. In one embodiment, the factor is TFPI. In another embodiment, the factor is SERPINE2 and/or Tfpi. In another embodiment, a cytokine, including SCF and/or Tpo, is administered.

Another embodiment provides for the use of the cells and/or factors described herein in medical therapy or the use of the cells and/or factors described herein to prepare a medicament to use in medical therapy.

### BRIEF DESCRIPTION OF THE FIGURES

FIG. 1 depicts isolation of Lineage negative BM. A) Total BM stained with PE conjugated antibodies against lineage markers and anti-c-kit APC. B) BM cells stained after lineage depletion using Stem Cell Technologies Hematopoietic Progenitor Enrichment Selection Kit with PE conjugated antibodies against lineage markers and anti-c-kit APC. C) KLS population in Total BM and D) lineage negative BM. Cells were stained with PE conjugated antibodies against lineage markers and anti-c-kit APC and anti-Sca-1 FITC.

FIG. 2 presents a schematic model of transwell culture.

FIG. 3 depicts total Cell and CFC Expansion of Lin− BM cells in Contact and Non-contact Stromal Cultures. 10⁴ Lin− BM cells were cultured either in direct contact with or in transwells above UG26-1B6, EL08-1D2 or AFT024 cells that had been irradiated at 2,500 cGy. After 3 weeks of culture, A) total cell number was enumerated to determine cell expansion. Trypan blue was used to distinguish the live cells from the irradiated stromal feeder and B) progeny were plated in CFC assay. CFC were enumerated at day 12. The data represents 3 individual experiments±standard deviation. % CFC recovery=(#CFC generated by the progeny of 10⁴ Lin− BM cells recovered after 3 weeks of culture/#CFC/10⁴ fresh Lin− BM cells)×100.

FIG. 4 demonstrates that UG26-1B6 cells, but not AFT024 and EL08-1D2 can maintain HSC in non-contact cultures. A combination of 2×10⁵ CD45.2+ competitor BMMNC and 10⁴ fresh CD45.1+ Lin− BM cells, or 10⁴ CD45.1+ Lin− BM cells cultured for 3 weeks in contact with or in transwell above AFT024, UG26-1B6 or EL08-1D2 cells were transplanted in CD45.2+ recipients. Twelve to sixteen weeks after transplantation, PB was collected and analyzed by FACS for presence of CD45.2+ and CD45.1+ cells. Data points represent total CD45.1+ cells in the CD45.2+ recipient animals. The frequency of engraftment represents the number of mice that showed both >1% overall engraftment and evidence of multi-lineage engraftment versus the total number of mice transplanted.

FIG. 5 demonstrates that Lin− BM co-cultured in contact with all feeders and in transwells above UG26-1B6 cells have multi-lineage potential in competitive repopulation assays; however repopulation by progeny from EL08-1D2-contact cultures is skewed towards the T-lymphoid lineage. A) The FACS plots demonstrate multi-lineage engraftment (Gr-1/Mac-1+, B220+, and CD4/CD8+ cells) of one representative mouse from each group transplanted with cells co-cultured with stromal cells. Analysis was done on PB 3 months after transplantation. B) The figure demonstrates multilineage repopulation of all transplanted mice that were engrafted with donor-derived cells. Bars represent percent±standard deviation for myeloid, B-lymphoid and T-lymphoid cells within the CD45.1+ population.

FIG. 6. Addition of 25% UG26-1B6 cells to EL08-1D2 feeders enables maintenance of HSC cultured in transwells above the feeder. 2×10⁵ CD45.2+ BMMNC were competed against 10⁴ CD45.1+ Lin− BM cells cultured for 3 weeks in transwells above a mixture of UG26-1B6 and EL08-1D2 cells in CD45.2+ recipients. Twelve to sixteen weeks after transplantation, PB was collected and analyzed by FACS for presence of CD45.2+ and CD45.1+ cells. Data points represent total CD45.1+ cells in the PB of CD45.2+ recipient animals. The frequency of engraftment represents the number of mice that showed both >1% engraftment, and multi-lineage engraftment versus the total number of mice transplanted.

FIG. 7. Cellular component of genes highly expressed in UG26-1B6 compared to EL08-1D2. RNA was extracted from irradiated UG26-1B6 and EL08-1D2 cells cultured for 7 days with Lin− BM cells in transwells above the feeders. Labeled cRNA was hybridized to Affymetrix mouse 430 2.0 chips in triplicate. For differentially expressed genes (see Examples: Material and Methods), the Ingenuity database (www.ingenuity.com) was used to categorize genes by cellular component.

FIG. 8. Effects of Tfpi, SerpinE2, and Galectin on HSC. A&B) 50 KLS cells per well were cultured in serum-free media with 50 ng/ml SCF and 100 ng/ml Tpo with or without 100 ng/ml Tfpi, 5 μg/ml SerpinE2, or 5 μg/ml galectin for 5 days. A) Fold expansion at day 5 from three separate isolations of KLS cells (mean±standard deviation). B) Total number of CFC per 10,000 Day 0 KLS cells counted 10 days after cells were seeded. The combined data represents three individual experiments (mean±standard deviation. C&D) Fresh or the progeny of 200 CD45.1+ KLS cells, cultured with Tpo, SCF with or without Galectin, Tfpi or SerpinE2 were competed with 10⁵ CD45.2+ BMMNC and transplanted IV into lethally irradiated CD45.2+ recipients. PB was analyzed for CD45.1+ multilineage donor-derived engraftment at C) 4 weeks and D) 12-16 weeks. Each point represents recipients with >1% donor-derived engraftment that contributed to all three lineages (Myeloid, T-lymphoid, and B-lymphoid).

FIGS. 9A and 9B provide data regarding adhesion and migration experiments using Tfpi.

### DETAILED DESCRIPTION OF THE INVENTION

### Definitions

“A” or “an” means one or more than one.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning.

“Effective amount” generally means an amount which provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

“Treat,” “treating” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

“Subject” means a vertebrate, such as a mammal, such as a human. Mammals include, but are not limited to, humans, dogs, cats, horses, cows and pigs.

“Stem cell” refers to a cell which is an undifferentiated cell capable of (1) long term self-renewal, or the ability to generate at least one identical copy of the original cell, (2) differentiation at the single cell level into multiple, and in some instances only one, specialized cell type and/or (3) of in vivo functional regeneration of tissues. Stem cells are subclassified according to their developmental potential as totipotent, pluripotent, multipotent and oligo/unipotent.

“Self-renewal” refers to the ability to produce replicate daughter stem cells having differentiation potential that is identical to those from which they arose. A similar term used in this context is “proliferation.”

“Stem cell” means a cell that can undergo self-renewal (i.e., progeny with the same differentiation potential) and also produce progeny cells that are more restricted in differentiation potential. Within the context of the invention, a stem cell would also encompass a more differentiated cell that has dedifferentiated, for example, by nuclear transfer, by fusions with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

### Hematopoietic Stem Cells

Blood cell formation, also known as hematopioesis, is a hierarchical process by which the hematopoietic stem cells (HSCs) give rise to committed hematopoietic progenitor cells (HPCs) that are capable of generating the entire repertoire of mature blood cells over the lifetime of an organism. HSCs, the pinnacle of the hematopoietic hierarchy, are functionally defined by their capacity for self-renewal, to maintain or expand the stem cell pool; multi-lineage differentiation, to generate and/or regenerate the mature lympho-hematopoietic system; and ultimately to home to the appropriate microenvironment in vivo where, through self-renewal and multi-lineage differentiation, they can restore normal hematopoiesis in a myeloablated host. As HSC differentiate they give rise to committed hematopoietic progenitor cells with limited self-renewal capacity and an increasingly restricted lineage potential. The earliest HSC cell-fate decision involves differentiation into either a common lymphoid or a common myeloid progenitor (CLP and CMP, respectively), establishing the major lymphoid and myeloid divisions of lympho-hematopoiteic system. As the name implies, the CLP gives rise to the mature lymphoid B, T and NK cells; and the CMP gives rise to both megakaryocyte-erythrocyte progenitors (MEPs) and granylocyte-monocyte progenitors (GMPs) that further differentiate into the mature myeloid megakaryocytic, erythroid, granulocytic and monocytic lineages.

During human development hematopoiesis occurs in two distinct waves. The first wave, known as “primitive” hematopoiesis, arises in the extra-embryonic yolk sac beginning around 16 days of gestation and primarily gives rise to primitive nucleated red blood cells to facilitate oxygen delivery (RBCs). A subsequent “definitive” wave of hematopoiesis begins around 27 days of gestation with the appearance of long-term repopulating HSC capable of lympo-myeloid differentiation within the aorta/gonads/mesonephros (AGM) region of the embryo proper. These definitive HSCs migrate from the AGM and seed the fetal liver (FL), which is the major site of fetal hematopoiesis from six to 22 weeks. After 22 weeks, the FL HSC continue migrating, ultimately finding their way to the bone marrow microenvironment, the primary site of hematopoiesis into adulthood. While HSC from ontogenically distinct origins have unique functional attributes, they share the defining self-renewal and multi-lineage differentiation characteristics.

Throughout ontogeny, normal hematopoiesis requires a tightly regulated balance between HSC self-renewal and lineage differentiation to ensure the adequate production of mature blood cells while maintaining the HSC pool. The consequences of dysregulating this balance are exemplified in nature by aplastic anemia on one extreme, an exhaustion of the HSC pool resulting from lineage differentiation without adequate self-renewal, and on the other extreme by hematologic malignancies, clonal expansion of immature blood cells as a result of enhanced self-renewal of the stem cell itself, or from a more committed cell without terminal differentiation. The balance of HSC self-renewal and differentiation is influenced by the convergence of intrinsic cellular signals and extrinsic micro-environmental cues from the surrounding stem cell niche, but the specific signals that regulate HSC cell-fate decisions are only poorly understood.

The complexities of HSC development and the regulation of cell-fate decisions are of particular interest, as hematopoietic cell transplantation (HCT), also known as bone marrow transplantation (BMT), has been effectively used as a clinical therapy for almost four decades. HCT represents a mainstay of therapy for numerous hematologic and non-hematologic malignancies as well as non-malignant blood diseases, and HSCs also represent a vector for gene therapy applications. Standard HCT protocols involve ablation of a patient's failing or malignant hematopoietic system using radiation and/or chemotherapy followed by transplant of either bone marrow (BM) or granulocyte colony-stimulating factor (G-CSF) mobilized peripheral blood (MPB) from either the patient (autologous HCT) or a suitably histocompatibility antigen-matched (also know as HLA-matched) donor (allogeneic HCT) to restore normal hematopoiesis. The limited availability of suitably matched BM or MPB donors represents a major obstacle for HCT. As a result of this limitation, increasing attention has shifted toward developing alternative cell sources for HCT, and umbilical cord blood (UCB) has emerged as the primary candidate. UCB represents an ideal cell source for HCT for two main reasons. First, on a per cell basis HSCs from UCB have a greater in vitro proliferative potential and increased in vivo engraftment potential compared to HSCs from ontogenetically later sources (e.g. BM and MPB). Second, there is a lower than expected incidence of graft-versus-host disease compared to BM or MPB, a life-threatening complication following HCT, following UCB transplantation, likely due to the naïveté of the developing immune system. In spite of these advantages, the clinical utility of UCB as a graft is limited by the low and fixed number of HSCs present in available UCB units. Additionally, quiescent HSCs from UCB and other sources are refractory to gene delivery using standard oncoretroviral vectors that require cell division for efficient integration into the genomic DNA. Therefore, a major focus of experimental hematology is to develop conditions suitable for the ex vivo expansion of HSCs.

The functional analysis on specific hematopoietic cell populations has been facilitated by the development of both monoclonal antibodies that recognize cell-surface molecules expressed on distinct subsets of hematopoietic cells and high-speed fluorescence activated cell sorting (FACS) to prospectively isolate pure cell populations. Classically, selection for CD34⁺ cells has provided an effective means to enrich for in vivo repopulating human HSCs, but the recent identification of CD34⁻ in vivo repopulating human HSCs and evidence that CD34 expression is reversible on HSCs imply that this strategy for HSC enrichment isolates only a portion of human HSCs. Furthermore, fewer than 0.05% of CD34⁺ UCB cells are capable of long-term repopulation of the bone marrow of myeloablated xenogenic recipients, the gold standard assay for the enumeration of human HSCs. The purity of human HSCs can be further increased by isolating CD34⁺ cells that do not express CD38 and lineage-specific surface antigens, so called CD34⁺ CD38⁻Lineage(Lin)⁻ cells, which comprise between 0.05-0.10% of UCB mononuclear cells. To date, the CD34⁺ CD38⁻Lin⁻ fraction of UCB represents one of the purest populations of long-term in vivo repopulating HSCs, but it remains considerably heterogeneous as it contains fewer than 0.2% repopulating HSCs. Therefore, even highly-purified, phenotypically-identical subsets of human UCB cells contain considerable functional heterogeneity. In lieu of the ability to prospectively identify and isolate pure populations of human HSCs and HPCs, numerous in vitro and in vivo functional assays have been developed to measure the quantity and quality of human HSCs and HPCs contained within heterogeneous cell populations in a retrospective fashion.

In Vitro Analysis of Human HSCs and HPCs

While in vivo assays provide the most physiologically relevant context for the functional characterization of human hematopoietic cells, the ability to analyze the functional attributes of limiting numbers of cells, or even single cells, under defined conditions makes in vitro assays an invaluable tool. The developmental continuum of hematopoiesis begins with HSCs, cells with tremendous self-renewal and multi-lineage differentiation potential, that give rise to committed HPCs, endowed with an increasingly limited capacity for self-renewal and restricted lineage differentiation potential, that ultimately give rise to terminally differentiated blood cells, incapable of either self-renewal or further differentiation. Therefore, hematopoietic cells can be characterized and classified in vitro based on their capacity for self-renewal and the lineage diversity of their progeny. Short-term in vitro assays, as their name implies, are designed to enumerate lineage-restricted HPCs that have a very limited self-renewal potential and are capable of differentiation into mature hematopoietic cells within two to three weeks in response to appropriate cytokine stimulation after minimal HPC division. Short-term in vitro assays specifically detect lineage-restricted myeloid HPCs, but not HSCs or primitive HPC subsets that are incapable of generating colonies in these protracted two to three week cultures. The standard assay for the enumeration of lineage-restricted HPCs is the colony-forming cell (CFC) assay in which cells are suspended in a semi-solid methylcellulose matrix culture system containing granulocyte colony stimulating factor (G-CSF), granulocyte-monocyte colony stimulating factor (GM-CSF), stem cell factor (SCF), interleukin 3 (1L3), and erythropoietin (Epo) to promote myelo-erythroid differentiation. Under these conditions, lineage-restricted HPCs give rise to distinct colonies, so called colony-forming units in culture (CFU-C), that can be enumerated to determine CFU-C frequency. The resulting colonies can be categorized based on colony size, morphology, and cell composition into colony-forming unit granulocyte-monocyte (CFU-GM), colony-forming unit erythroid (CFU-E), burst-forming unit erythroid (BFU-E), and multi-lineage colonies represented by colony-forming unit mix (CFU-mix), comprised of both granulocyte-monocyte and erythroid progeny, or colony-forming unit granulocyte-erythrocyte-monocyte-megakaryocyte (CFU-GEMM) comprised granulocyte, monocyte, erythroid and megakaryocyte progeny. Additionally, the relative maturity of CFU-Cs can be assessed using the CFC assay. Unlike more mature CFU-Cs, more primitive CFU-Cs are larger, in most cases containing multiple differentiated cell types, and can generate secondary colonies when re-plated in fresh CFC assays (secondary CFU-Cs), a reflection of their increased proliferative potential. While a majority of short-term hematopoietic progenitor assays exclusively detect the presence of myeloid progenitors, clonogenic in vitro assays to detect the presence of a short-term myeloid/lymphoid progenitors capable of generating B cells, T cells, and myeloid cells from murine fetal liver and the presence of murine pre-B cell precursors have also been described.

In vitro assays have also been developed to evaluate the presence of more primitive subsets of hematopoietic progenitor cells that likely represent an intermediate level of maturity between long-term HSC and short-term CFU-Cs, so called blast colony-forming cells (blast-CFCs) and high proliferative potential-colony-forming cells (HPP-CFCs). Blast-CFCs represent a rare population of cells that are capable of forming colonies of 40-1,000 small retractile cells that lack signs of terminal differentiation in clonogenic assays and are capable of giving rise to multi-lineage CFU-GEMM colonies in secondary CFC assays with a near 100% re-plating efficiency. HPP-CFC represent another class of primitive hematopoietic progenitors that are capable of generating large (>0.5 mm) monocyte/macrophage colonies after 14 days in semi-solid medium. Characteristic of primitive hematopoietic cell populations, both blast-CFCs and HPP-CFC share a relative resistance to 5-FU toxicity, a reflection of their quiescent nature relative to more mature CFU-Cs.

Primitive HPCs and presumed HSCs were originally detected in vitro in heterogeneous long-term bone marrow culture (LTBMC) capable of sustained production of hematopoietic progenitor cells in culture. Subsequently, conditions were defined that enabled the enumeration of primitive cells that are incapable of giving rise to colonies in short-term CFC, blast-CFC, or HPP-CFC assays, but generate clonogenic progenitor cells after extended culture periods (˜5 weeks) that support the maturation and subsequent readout. One such long-term in vitro assay is the cobblestone-forming cell (CAFC) assay in which cells are maintained in liquid culture in contact with a monolayer of irradiated, or otherwise mitotically inactivated, stromal cells that partially recapitulate the complex in vivo bone marrow microenvironment to support HSC maturation. Long-term CAFCs are cells capable of generating a cobblestone-like colony of flattened cells in close association with the supporting stromal layer after five or more weeks in culture, signifying the long-term survival and proliferative potential of these primitive cells, defining characteristics of primitive hematopoietic cells. Another long-term in vitro assay is the long-term culture initiating cell (LTC-IC) assay that also assesses the long-term survival and proliferative potential of primitive hematopoietic cells after 35-60 days (standard LTC-IC) or 60-100 days (extended (e)LTC-IC) in liquid co-cultures containing supportive stromal cells. In contrast to the CAFC assay, the LTC-IC assay read-out is the ability of cells maintained for >5 weeks in liquid culture to generate one or more secondary CFU-Cs when placed in CFC conditions. LTC-IC assays are commonly performed using bulk cell populations to provide a crude measure of the total number of CFU-C generated per input cell. However, LTC-IC assays performed in this way cannot enumerate input LTC-IC frequencies, as a single LTC-IC can give rise to multiple CFU-C. Although it has been postulated that LTC-IC generate on average 4 CFU-Cs, differing culture conditions may affect the number of secondary CFU-Cs per input LTC-IC. Hence to enumerate the exact input number of LTC-ICs, standard limiting dilution experiments and Poisson statistics are used. Combining bulk and limiting dilution experiments, the number of CFU-C per input LTC-IC can be determined, providing both LTC-IC frequency and a measure of the generative potential of LTC-IC. While it has been speculated that LTC-IC represent a more primitive subset of CAFC that retain clonogenic potential after long-term culture, there is currently little data to support this hypothesis. The validity of the use of in vitro assays to enumerate cells with primitive hematopoietic function is supported by the strong correlation between the frequency of murine LTC-IC and CAFC with in vivo repopulating cells and data demonstrating a moderate to high correlation of human LTC-IC frequencies and in vivo repopulation potential for freshly isolated hematopoietic cells. However, human cells capable of in vivo repopulation in xenogenic transplant models have a much lower frequency than in vitro LTC-IC, and they are not maintained to the same degree as LTC-IC during ex vivo expansion cultures. These differences likely result from differences in the efficiency of detecting primitive cells using in vitro versus in vivo assays as well as differences in the functional requirements for in vitro defined HSC versus in vivo defined HSC.

HSCs are endowed with the capacity to generate not only myeloid progeny, but also lymphoid progeny. Thus, a major shortcoming of long-term in vitro HSC assays, such as the LTC-IC assay, is that they exclusively assess the generation of myeloid progeny, and thus may overestimate true HSC frequencies. The first in vitro system developed to evaluate the long-term development of lymphoid progeny was the Whitlock-Witte culture system, in which LTBMC conditions were optimized to support the development of heterogeneous populations of pre-B, immature B, and mature B lymphocytes. To more closely recapitulate the in vivo differentiation programs of HSCs in vitro, assays that assess both long-term myeloid and lymphoid potential of human hematopoietic cells were subsequently developed. In the lymphoid-myeloid “switch culture” single cells are seeded on S17 stromal cells that primarily support the generation of CD 19⁺ B-cell progenitors, and following proliferation during a primary expansion culture, the progeny of the input cell are evaluated for myeloid potential, determined by expression of CD33 and/or CFC formation, in secondary switch cultures. The myeloid-lymphoid initiating cell (ML-IC) assay also enumerates single cells that are capable of giving rise to at least one long-term myeloid progenitor and one long-term lymphoid progenitor in vitro. Neither the switch culture or ML-IC assay is suitable for evaluating the T lymphoid potential of input progenitor cells, as in vitro T cell development requires complex interactions with thymic stromal cells in three dimensional fetal thymic organ cultures (FTOC) or co-culture with specialized transgenic stromal cells capable of supporting T lymphogenesis. Despite this caveat, the multi-potent progenitor detected in the switch culture and ML-IC assays likely represents a more primitive progenitor than LTC-IC, and more closely resembles the potential of true HSCs. Furthermore, input of single cells in the ML-IC assay enables the determination of both frequency and generative potential of input ML-IC in a single assay. Therefore, the switch culture and ML-IC assays represent the most robust in vitro human HSC assays.

In Vivo Analysis of Human HSCs and HPCs

In vitro assays, that evaluate the self-renewal and multi-lineage differentiation potential of input cells, have been an invaluable tool for the qualitative and quantitative analysis of human hematopoietic cells; however, true HSC and HPC must not only self-renew and differentiate in vitro, but must also be able to home to the appropriate microenvironment in vivo to restore normal hematopoiesis in a myeloablated recipient. While there are advantages to the defined conditions that are attainable in vitro, it is not possible to accurately recapitulate the complex three dimensional cell-cell and cell-matrix interactions of the bone marrow microenvironment in a two dimensional culture system. Therefore, in vivo assays provide the most rigorous systems for the evaluation of HSC and HPC function. While in vivo transplant of human HSCs into human recipients would provide the most physiologically relevant assay of HSC function, such experiments are not tenable on ethical grounds. Therefore, in vivo assays for human HSCs rely on xenogenic recipients to evaluate their in vivo potential. However, xenogeneic transplants are complicated by immune-mediated rejection of the transplanted human cells by recipient animals. As a consequence, in vivo assays have been developed using various immunocompromised animal models.

One of the most well established xenogeneic transplant recipients for human HSC is the nonobese diabetic-severe combined immunodeficiency (NOD-SCID) mouse, that has defects in innate immunity and harbors a mutation in the Prkdc gene, rendering it incapable of generating mature B and T cells and thus unable to effectively reject transplanted human cells. Long-term engraftment of human hematopoietic cells in the bone marrow of NOD-SCID mice, so called SCID-repopulating cells (SRC), requires the transplantation of hundreds to thousands of human cells for successful engraftment, and thus is not a clonal assay. As such, the presence of long-term repopulating HSCs with self-renewal and multi-lineage differentiation potential is inferred from the ability of engrafted cells to generate both myeloid and lymphoid lineages in primary transplant recipients for greater than six to eight weeks, the presumed lifespan of primitive HPCs. The clonal contributions of multi-lineage, long-term SRC in engrafted recipients can be established by retrovirally marking SRCs prior to transplant. In this way, integration of pro-viral DNA into the SRC genome serves as a molecular signature to identify the clonally related progeny of single SRCs, and thereby directly establishes the long-term, multi-lineage differentation potential of single cells. Long-term self-renewal and multi-lineage differentiation potential of SRCs can be more rigorously evaluated by assessing engraftment of SRCs after extended periods of time (i.e. 12-14 weeks), or more robustly by performing serial transplants of SRCs into successive recipients. While retroviral marking of SRCs provides some information regarding the numbers of SRCs in a given cell population, the frequency of SRCs is more commonly determined by transplanting cells in limiting dilutions.

While the classic NOD-SCID xenotransplant model provides a robust assay for multi-lineage human HSC engraftment, its utility to investigate transplant immunology has been limited by the failure of human lymphoid progenitors to efficiently migrate to the murine thymic microenvironment to generate mature T cells. Therefore, alternative models have been exploited for studying the in vivo development of adaptive immunity. The SCID-hu thymus mouse contains fragments of human thymic tissue that are transplanted under the kidney capsule of SCID mice to provide a xenograft microenvironment for the development of human thymopoiesis. The Rag2^{-/-}γ_{c}^{-/-} mouse has a deletion of both the Rag2 DNA recombinase activity, precluding the development of mature B and T cells, and lacks the IL2 receptor γ common chain, required for IL2, IL4, IL7, IL9 and IL15 signaling, resulting in profound adaptive and innate immunodeficiency. This double knock-out model facilitates the development of a functional adaptive immune system from human cells transplanted into the fetal liver. The NOD-SCID-γ_{c}^{-/-} mouse, harboring a γ common chain deletion on the NOD-SCID background has also proven an effective model to study human immunity. Therefore, selecting a suitable model for xenotransplant of human stem and progenitor cells is dictated by the particular progenitors and progeny that are of interest.

### Methods and Compositions of the Invention

The methods of the invention maintains stem cells, more particularly HSCs, in culture. Such culture methods can be used to expand these stem cells.

Culture methods of the invention can comprise an overexpression of the genes disclosed herein or a part (e.g., a biologically active fragment) or derivative thereof, or the addition of the protein the gene encodes to the culture system.

The invention also includes the development of small molecules/factors that increase HSC self-renewal in vitro and in vivo.

### Stem Cells

The present invention can be practiced using stem cells (e.g., HSC) of vertebrate species, such as humans, non-human primates, domestic animals, livestock, and other non-human mammals.

In an embodiment of the invention, supplements to keep maintain/expand stem cells, more particularly HSCs, include those cellular factors disclosed herein or components thereof that allow maintenance/expansion of said stem cells. This may be indicated by the number of stem cells present in a given sample.

The present invention also provides methods wherein constitutive overexpression of the genes described herein increases the maintenance/expansion of KLS cells in vitro.

### Cell Culture

In general, cells useful for the invention can be maintained and expanded in culture medium that is available to and well-known in the art. Such media include, but are not limited to, Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®, and serum-free medium for culture and expansion of hematopoietic cells SFEM®. Many media are also available as low-glucose formulations, with or without sodium pyruvate.

Also contemplated in the present invention is supplementation of cell culture medium with mammalian sera. Sera often contain cellular factors and components for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements and bovine embryonic fluid. It is understood that sera can be heat-inactivated at 55-65° C. if deemed necessary to inactivate components of the complement cascade.

Additional supplements also can be used advantageously to supply the cells with the trace elements for optimal growth and expansion. Such supplements include insulin, transferrin, sodium selenium and combinations thereof. These components can be included in a salt solution such as, but not limited to, Hanks' Balanced Salt Solution® (HBSS), Earle's Salt Solution®, antioxidant supplements, MCDB-201® supplements, phosphate buffered saline (PBS), ascorbic acid and ascorbic acid-2-phosphate, as well as additional amino acids. Many cell culture media already contain amino acids, however, some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

Hormones also can be advantageously used in the cell cultures of the present invention and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine and L-thyronine.

Lipids and lipid carriers also can be used to supplement cell culture media, depending on the type of cell and the fate of the differentiated cell. Such lipids and carriers can include, but are not limited to, cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin and oleic acid unconjugated and conjugated to albumin, among others.

Also contemplated in the present invention is the use of feeder cell layers. Feeder cells are used to support the growth of fastidious cultured cells, such as ES cells. Feeder cells are normal cells that have been inactivated by γ-irradiation. In culture, the feeder layer serves as a basal layer for other cells and supplies cellular factors without further growth or division of their own (Lim, J. W. and Bodnar, A., 2002). Examples of feeder layer cells are typically human diploid lung cells, mouse embryonic fibroblasts and Swiss mouse embryonic fibroblasts, but can be any post-mitotic cell that is capable of supplying cellular components and factors that are advantageous in allowing optimal growth, viability and expansion of stem cells. In many cases, feeder cell layers are not necessary to keep ES cells in an undifferentiated, proliferative state, as leukemia inhibitory factor (LIF) has anti-differentiation properties. Therefore, supplementation with LIF can be used to maintain cells in an undifferentiated state.

Cells may be cultured in low-serum or serum-free culture medium. Serum-free medium used to culture cells is described in, for example, U.S. Pat. No. 7,015,037. Many cells have been grown in serum-free or low-serum medium. For example, the medium can be supplemented with one or more growth factors. Commonly used growth factors include, but are not limited to, bone morphogenic protein, basis fibroblast growth factor, platelet-derived growth factor and epidermal growth factor, Stem cell factor, thrombopoietine, Flt3Ligand and 11-3. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference herein for teaching growing cells in serum-free medium.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. One embodiment of the present invention utilizes fibronectin. Hematopoietic stem cells can also be cultured in low attachment flasks such as but not limited to Corning Low attachment plates.

Once established in culture, cells can be used fresh or frozen and stored as frozen stocks, using, for example, DMEM with 40% FCS and 10% DMSO. Other methods for preparing frozen stocks for cultured cells also are available to those skilled in the art.

Methods of identifying and subsequently separating differentiated cells from their undifferentiated counterparts can be carried out by methods well known in the art. Cells that have been induced to inhibit differentiation using methods of the present invention can be identified by selectively culturing cells under conditions whereby undifferentiated cells have a specific phenotype identifiable by FACS. Similarly, differentiated cells can be identified by morphological changes and characteristics that are not present on their undifferentiated counterparts, such as cell size and the complexity of intracellular organelle distribution. Also contemplated are methods of identifying differentiated cells by their expression of specific cell-surface markers such as cellular receptors and transmembrane proteins. Monoclonal antibodies against these cell-surface markers can be used to identify differentiated cells. Detection of these cells can be achieved through fluorescence activated cell sorting (FACS) and enzyme-linked immunosorbent assay (ELISA). From the standpoint of transcriptional upregulation of specific genes, differentiated cells often display levels of gene expression that are different from undifferentiated cells. Reverse-transcription polymerase chain reaction, or RT-PCR, also can be used to monitor changes in gene expression in response to differentiation. Whole genome analysis using microarray technology also can be used to identify differentiated cells.

Accordingly, once differentiated cells are identified, they can be separated from their undifferentiated counterparts, if necessary. The methods of identification detailed above also provide methods of separation, such as FACS, preferential cell culture methods, ELISA, magnetic beads and combinations thereof. One embodiment of the present invention comtemplates the use of FACS to identify and separate cells based on cell-surface antigen expression.

Pharmaceutical Formulations

In certain embodiments, the purified cell populations, proteins described herein, biologically active fragments or derivatives thereof or small molecules are present within a composition adapted for and suitable for delivery, i.e., physiologically compatible. Accordingly, compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

In other embodiments, the purified cell populations are present within a composition adapted for or suitable for freezing or storage.

In many embodiments the purity of the cells for administration to a subject is about 100%. In other embodiments it is about 95% to about 100%. In some embodiments it is about 85% to about 95%. Particularly in the case of admixtures with other cells, the percentage can be about 10%-15%, about 15%-20%, about 20%-25%, about 25%-30%, about 30%-35%, about 35%-40%, about 40%-45%, about 45%-50%, about 60%-70%, about 70%-80%, about 80%-90%, or about 90%-95%. Or isolation/purity can be expressed in terms of cell doublings where the cells have undergone, for example, about 10-20, about 20-30, about 30-40, about 40-50 or more cell doublings.

The numbers of cells in a given volume can be determined by well known and routine procedures and instrumentation. The percentage of the cells in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

The choice of formulation for administering the compositions of the invention for a given application will depend on a variety of factors. Prominent among these will be the species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of administration and the nature of the particular dosage form.

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic cells. Various embodiments of the invention comprise measures to increase cell survival and/or to overcome problems posed by barriers to seeding and/or growth.

Final formulations of the aqueous suspension of cells/medium, protein and/or small molecules will typically involve adjusting the ionic strength of the suspension to isotonicity (i.e., about 0.1 to 0.2) and to physiological pH (i.e., about pH 6.8 to 7.5). The final formulation will also typically contain a fluid lubricant, such as maltose, which must be tolerated by the body. Exemplary lubricant components include glycerol, glycogen, maltose and the like. Organic polymer base materials, such as polyethylene glycol and hyaluronic acid as well as non-fibrillar collagen, such as succinylated collagen, can also act as lubricants. Such lubricants are generally used to improve the injectability, intrudability and dispersion of the injected material at the site of injection and to decrease the amount of spiking by modifying the viscosity of the compositions. This final formulation is by definition the cells, protein described herein, biologically active fragments or derivatives thereof or small molecules in a pharmaceutically acceptable carrier.

The compositions are subsequently placed in a syringe or other injection apparatus for precise placement at the preselected site. The term “injectable” means the formulation can be dispensed from syringes having a gauge as low as 25 under normal conditions under normal pressure without substantial spiking Spiking can cause the composition to ooze from the syringe rather than be injected into the tissue. For this precise placement, needles as fine as 27 gauge (200μ I.D.) or even 30 gauge (150μ I.D.) are desirable. The maximum particle size that can be extruded through such needles will be a complex function of at least the following: particle maximum dimension, particle aspect ratio (length:width), particle rigidity, surface roughness of particles and related factors affecting particle:particle adhesion, the viscoelastic properties of the suspending fluid, and the rate of flow through the needle. Rigid spherical beads suspended in a Newtonian fluid represent the simplest case, while fibrous or branched particles in a viscoelastic fluid are likely to be more complex.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

A pharmaceutically acceptable preservative or stabilizer can be employed to increase the life of cell/medium compositions. If such preservatives are included, it is well within the purview of the skilled artisan to select compositions that will not affect the viability or efficacy of the cells.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells/medium, protein or small molecules utilized in practicing the present invention in the required amount of the appropriate solvent with various amounts of the other ingredients, as desired.

In some embodiments, cells/medium, protein or small molecules are formulated in a unit dosage injectable form, such as a solution, suspension, or emulsion. Pharmaceutical formulations suitable for injection of cells/medium, protein or small molecules typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells, protein or small molecules and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %.

In some embodiments stem cells, protein and/or small molecules are encapsulated for administration, particularly where encapsulation enhances the effectiveness of the therapy, or provides advantages in handling and/or shelf life. Also, encapsulation in some embodiments provides a barrier to a subject's immune system.

A wide variety of materials may be used in various embodiments for microencapsulation. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation that may be used for administration are known to those of skill in the art and are described, for example, in Chang, P., et al., 1999; Matthew, H. W., et al., 1991; Yanagi, K., et al., 1989; Cai Z. H., et al., 1988; Chang, T.M., 1992 and in U.S. Pat. No. 5,639,275 (which, for example, describes a biocompatible capsule for long-term maintenance of cells that stably express biologically active molecules). Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference.

Certain embodiments incorporate cells, protein and/or small molecules into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, cells, protein and/or small molecules may be incorporated in the interstices of a three-dimensional gel. A large polymer or gel, typically, will be surgically implanted. A polymer or gel that can be formulated in small enough particles or fibers can be administered by other common, more convenient, non-surgical routes.

Dosing

Compositions (e.g., compositions containing cells, small molecules, protein or fragments or derivatives thereof) can be administered in dosages and by techniques well known to those skilled in the medical and veterinary arts taking into consideration such factors as the age, sex, weight, and condition of the particular patient, and the formulation that will be administered (e.g., solid vs. liquid). Doses for humans or other mammals can be determined without undue experimentation by the skilled artisan, from this disclosure, the documents cited herein, and the knowledge in the art.

The dose of cells/medium, protein or small molecules appropriate to be used in accordance with various embodiments of the invention will depend on numerous factors. It may vary considerably for different circumstances. The parameters that will determine optimal doses to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration. Finally, the determination of optimal dosing necessarily will provide an effective dose that is neither below the threshold of maximal beneficial effect nor above the threshold where the deleterious effects associated with the dose outweighs the advantages of the increased dose.

The optimal dose of cells for some embodiments will be in the range of doses used for autologous, mononuclear bone marrow transplantation. For fairly pure preparations of cells, optimal doses in various embodiments will range from about 10⁴ to about 10⁸ cells/kg of recipient mass per administration. In some embodiments the optimal dose per administration will be between about 10⁵ to about 10⁷ cells/kg. In many embodiments the optimal dose per administration will be about 5×10⁵ to about 5×10⁶ cells/kg. By way of reference, higher doses in the foregoing are analogous to the doses of nucleated cells used in autologous mononuclear bone marrow transplantation. Some of the lower doses are analogous to the number of CD34⁺ cells/kg used in autologous mononuclear bone marrow transplantation.

It is to be appreciated that a single dose may be delivered all at once, fractionally, or continuously over a period of time. The entire dose also may be delivered to a single location or spread fractionally over several locations.

In various embodiments, cells/medium, protein or small molecules may be administered in an initial dose, and thereafter maintained by further administration. Cells/medium, protein or small molecules may be administered by one method initially, and thereafter administered by the same method or one or more different methods. The levels can be maintained by the ongoing administration of the cells/medium, protein or small molecules. Various embodiments administer the cells/medium, protein or small molecules either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regimens can be ascertained by the skilled artisan, from this disclosure, the documents cited herein, and the knowledge in the art.

The dose, frequency, and duration of treatment will depend on many factors, including the nature of the disease, the subject, and other therapies that may be administered. Accordingly, a wide variety of regimens may be used to administer the cells/medium, protein or small molecules.

In some embodiments cells/medium, protein or small molecules are administered to a subject in one dose. In others cells/medium, protein or small molecules are administered to a subject in a series of two or more doses in succession. In some other embodiments wherein cells/medium, protein or small molecules are administered in a single dose, in two doses, and/or more than two doses, the doses may be the same or different, and they are administered with equal or with unequal intervals between them.

Cells/medium, protein or small molecules may be administered in many frequencies over a wide range of times. In some embodiments, they are administered over a period of less than one day. In other embodiment they are administered over two, three, four, five, or six days. In some embodiments they are administered one or more times per week, over a period of weeks. In other embodiments they are administered over a period of weeks for one to several months. In various embodiments they may be administered over a period of months. In others they may be administered over a period of one or more years. Generally lengths of treatment will be proportional to the length of the disease process, the effectiveness of the therapies being applied, and the condition and response of the subject being treated.

Additionally, proteins described herein in the examples, a biologically active fragment or derivative thereof or a small molecule can be administered to a subject in need thereof according to any formulations/regimens available to those of skill in the art including those discussed above for cells. For example, in certain embodiments medium or other suitable solution can comprise small molecules, protein or biologically active fragments or derivatives thereof.

Uses for the Cells

(1) Transplantation

Subjects in need for a stem cell (e.g. a HSC) transplantation could benefit from the cells produced by the methods of this invention. Subjects in need for such a transplantation include subjects suffering from non malignant blood disorders, particularly immunodeficiencies (e.g. SCID, fanconi's anemia, severe aplastic anemia, congenital hemoglobinopathies, and metabolic storage diseases such as for example, Hurler's disease, Hunter's disease, mannosidosis, among others) or cancer, particularly hematological malignancies such as acute leukemia, chronic leukemia (myeloid and lymphoid), lymphoma (Hodgkin's and non-Hodgkin's), multiple myeloma, myelodysplastic syndrome, or non-hematological cancers such as breast carcinoma, colon carcinoma, neuroblastoma, and renal cell carcinoma.

(2) Pharmaceutical Testing

Pharmaceutical testing is moving more and more from in vivo experimentation to in vitro studies. Since the availability of large numbers of stem cells (e.g. HSC) is insufficient for such testing, the methods of this invention can be used to produce stem cells that could be used for screening for pharmaceutical compounds.

Accordingly, the cells of the invention can be used in such testing and screening methods.

(3) Development of Small Molecules

Small molecules can screened to determine if they have the capacity to maintain and/or expand stem cells, e.g., HSCs. These molecules can then be used in vitro and in vivo to incease, for example, stem cell (HSC) self-renewal.

Uses of the Protein/Nucleic Acid

One embodiment provides a method for maintaining/expanding stem cells comprising culturing stem cells in the presence of one or more of the proteins described herein that are added as a protein or biologically active fragment or derivative thereof.

Another embodiment provides a method of treatment comprising administering (as described in above in the section entitled Pharmaceutical Formulations or Dosing) one or more proteins described herein or DNA coding for those proteins, a biologically active fragment or derivative thereof or a small molecule that increases self-renewal of stem cells to a subject in need thereof so as to increase stem cells in the subject, e.g., HSCs. Subjects in need of this type of treatment include subjects suffering from (e.g., afflicted with) non malignant blood disorders, particularly immunodeficiencies (e.g. SCID, fanconi's anemia, severe aplastic anemia, or congenital hemoglobinopathies, or metabolic storage diseases, such as Hurler's disease, Hunter's disease, mannosidosis, among others) or cancer, particularly hematological malignancies, such as acute leukemia, chronic leukemia (myeloid or lymphoid), lymphoma (Hodgkin's or non-Hodgkin's), multiple myeloma, myelodysplastic syndrome, or non-hematological cancers such as breast carcinoma, colon carcinoma, neuroblastoma, or renal cell carcinoma.

Additionally, cytokines/growth factors can be administered. For example, exogenous factors (e.g., cytokines, differentiation factors and other factors) can be administered solely or prior to, after or concomitantly with the cells and/or proteins of the invention. For example, a form of concomitant administration would comprise combining a factor of interest in the culture media and/or pharmaceutically acceptable carrier prior to administration. Doses for administrations are variable, may include an initial administration followed by subsequent administrations; but nonetheless, can be ascertained by the skilled artisan, from this disclosure, the documents cited herein, and the knowledge in the art.

A parameter involved in the therapeutic use of cells is the quantity of cells needed to achieve an optimal effect. In current human studies of autologous mononuclear bone marrow cells, empirical doses ranging from 1 to 4×10⁷ cells have been used with encouraging results. However, different scenarios may require optimization of the amount of cells injected into a tissue of interest. Thus, the quantity of cells to be administered will vary for the subject being treated. In one embodiment, between 10⁴ to 10⁸, more preferably 10⁵ to 10⁷, and most preferably 3×10⁷ progenitorcells and optionally, 50 to 500 μg/kg per day of a cytokine can be administered to a human subject. However, the precise determination of what would be considered an effective dose may be based on factors individual to each patient, including their size, age, size tissue damage, and amount of time since the damage occurred. Therefore, dosages can be readily ascertained by those skilled in the art from this disclosure and the knowledge in the art.

Additionally, the skilled artisan can readily determine the amount of cells and optional additives, vehicles, and/or carrier in compositions to be administered in methods of the invention. Typically, any additives (in addition to the active cell(s) and/or cytokine(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

The present invention is additionally described by way of the following illustrative, non-limiting Examples that provide a better understanding of the present invention and of its many advantages.

### EXAMPLES

### Material and Methods For Examples

### Animals

Transplantation and HSC Isolation

8 to 10 week old C57BL/6J (CD45.2) female mice were used as recipient mice and B6.5JL-PTPRCA (CD45.1) male mice were used as donor mice. All mice were purchased from Jackson Laboratories (Bar Harbor, Me.). Mice were maintained at the University of Minnesota Research Animal Resources in specific pathogen free conditions or Katholieke Universiteit Leuven.

### Isolation of Bone Marrow Populations

Isolation of Lin− BM Cells

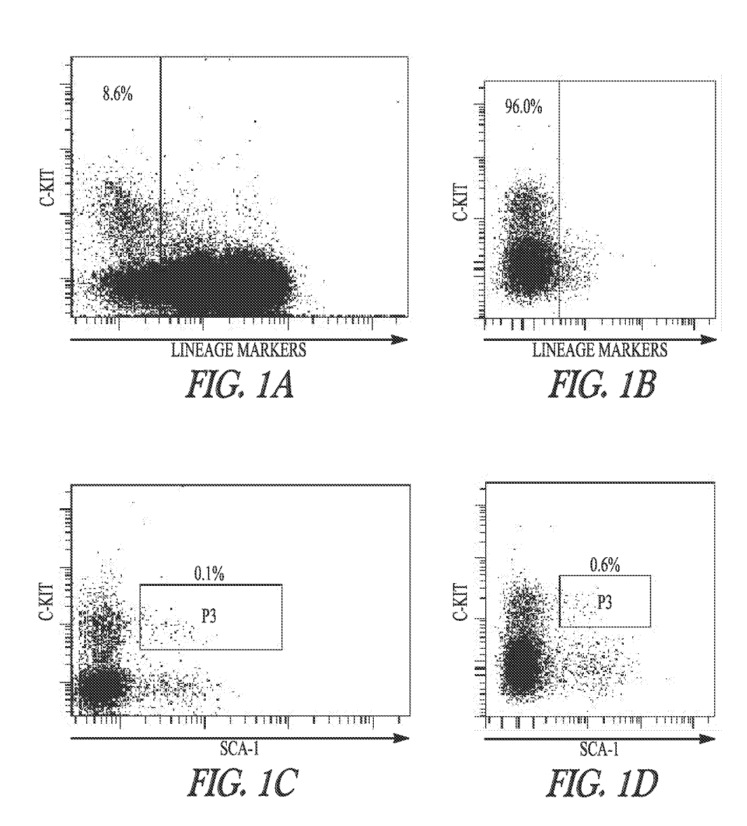

Femurs and tibia from mice were removed, BM flushed, and depleted of red blood cells by ammonium chloride (Stem Cell Technologies, Vancouver, BC, Canada). Lineage negative (Lin−) cells were obtained using the Stem Cell Technologies Lineage Negative Selection Kit (Stem Cell Technology) per manufacturer's protocol. (FIG. 1)

Isolation of KLS BM Cells

For KLS cell isolation, Lin− BM cells isolated as described above, were stained with the following antibodies: anti-ckit (2B8) APC, anti-Sca-1 (E13-161.7) PE and biotinylated antibodies against the lineage markers (Gr-1 (RB6-8C5), Mac-1 (M1/70), B220 (RA3-6B2), CD4 (H129.19), CD8 (53.6.7), Terr-119 (Ly-76)) followed by strepavidin FITC (BD Pharmingen, San Jose, Calif.). KLS cells were then sorted on a FacsVantage with Diva update.

Isolation of KLS/CD34− BM Cells

Isolation of KLS/CD34− BM Cells

Lin− BM cells were isolated as described above and stained with the following antibodies: anti-CD34 FITC (RAM34) (eBioscience, San Diego, Calif.), anti-ckit (2B8) APC, anti-Sca-1 (E13-161.7) PE and biotinylated antibodies against the lineage markers (Gr-1 (RB6-8C5), Mac-1 (M1/70), B220 (RA3-6B2), CD4 (H129.19), CD8 (53.6.7), Terr-119 (Ly-76)) followed by strepavidin peridinin chlorophyll (PerCP) (BD Pharmingen, San Jose, Calif.). KLS cells were then sorted on a FacsVantage with Diva update (BD Biosciences, San Jose, Calif.).

### Cell Culture

Stromal Cell Lines

UG26-1B6 and EL08-1D2 stromal cell lines were cultured as previously described [1]. The AFT024 fetal liver cell line was cultured as previously described [2, 3]. For stromal cell mixing experiments, UG26-1B6 cells were transduced with the MSCV-eGFP vector [4]. eGFP+UG26-1B6 cells were sorted using the FacsAria to a purity of 98+1%.

Stroma-Contact Cultures

Stromal cell lines were grown on 0.1% gelatin (Sigma, St. Louis, Mo.) in 6-well plates (Corning, Lowell, Mass.). Once cells reached confluence plates were irradiated at 20-25 Gy. 1 or 5×10⁴ Lin− BM cells were seeded in 3 ml of LTC-medium (Stem Cell Technologies, Vancouver, BC, Canada) with hydrocortisone (10⁻⁶ M, Stem Cell Technologies), and cultured for 3 weeks. Each week one half of the medium was removed and replaced with fresh medium. After 3 weeks, adherent and non-adherent cells were collected and assayed for presence of CFC and repopulating HSC.

Stroma-Non-Contact Cultures

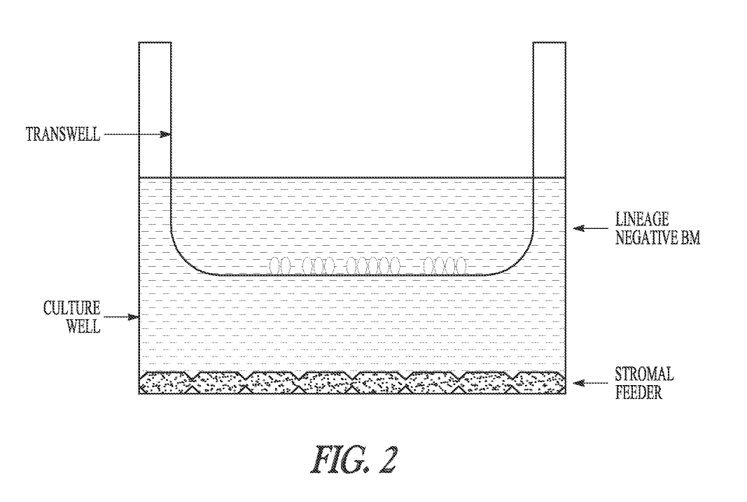

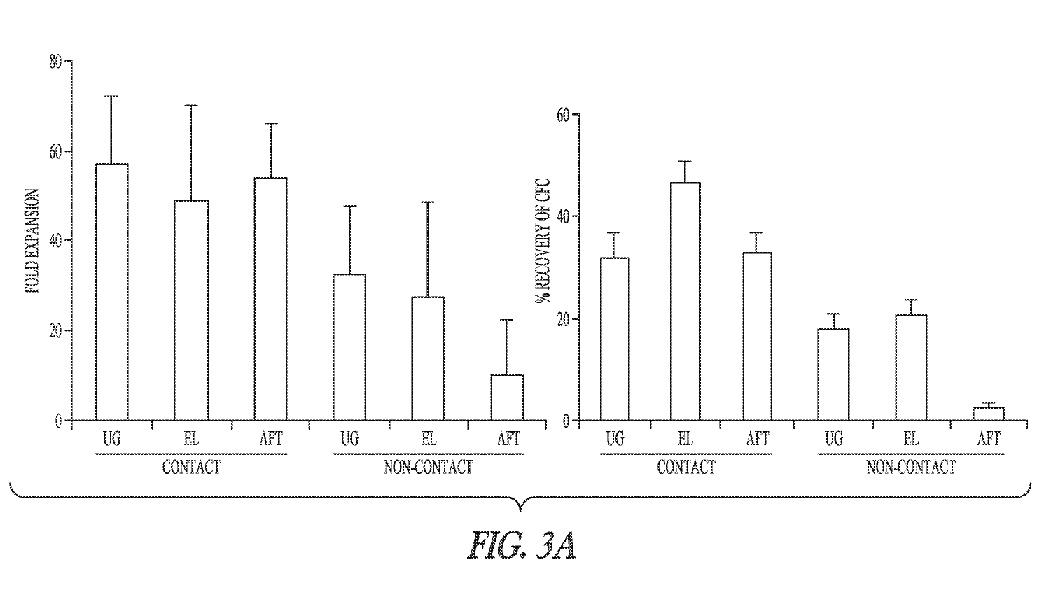

For non-contact cultures, stromal cell lines were grown on 0.1% gelatin (Sigma) in 6-well plates (Corning). Once cells reached confluence plates were irradiated at 20-25 Gy. Lin⁻ BM cells were plated in a 0.4 μm collagen coated transwell (Costar, location) placed above the feeders (FIG. 2). Each week, half of the medium was removed from beneath the transwell and fresh medium added to the transwell insert. After 3 weeks, the adherent feeder layer below the transwell and cells in the transwell were collected, and assayed for CFC and repopulating HSC. Some cultures were supplemented with 10 ng/ml recombinant mWnt5a, 1 μg/ml anti-Wnt5a, or 1 μg/ml anti IgG (R&D Systems, Minneapolis, Minn.).

In some studies the eGFP⁺ UG26-1B6 and EL08-1D2 feeders were mixed using the following ratios: 100% UG26-1B6/0% EL08-1D2, 75% UG26-1B6/25% EL08-1D2, 50% UG26-1B6/50% EL08-1D2, 25% UG26-1B6/75% EL08-1D2, or 0% UG26-1B6/100% EL08-1D2 cells. To confirm the relative ratios of cells present in the feeder, the percent eGFP⁺ cells were determined by both fluorescent microscopy and by flowcytometry.

KLS Stroma-Free/Serum-Free Cultures

For KLS stroma-free cultures, 50-200 KLS cells were plated per well of a 96-well U-bottom plate (BD Biosciences, San Jose, Mass.) in 100₁a1 of Stemspan (Stem Cell Technologies) supplemented with 100 ng/ml mTpo, 50 ng/ml mSCF, and/or 100 ng/ml mWnt5a, 100 ng/ml TFPI, 100 ng/ml SerpinE2, 5 μg/ml Galectin, 100 ng/ml BMP-4, 5 μg/ml Tsg, 5 μg/ml Chd (all from R&D Systems). Cells were cultured for 5 days at 37° C. with 5% CO₂. The culture system was adapted from Zhang, et. al. [5].

### Colony-Forming Cell (CFC) Assay

Fresh or culture progeny were plated in methylcellulose medium (M3234, Stem Cell Technologies) supplemented with 20 ng/ml mSCF, 10 ng/ml mIL-3, 10 ng/ml mIL-6 (all from R&D Systems), and 3 U/ml hEpo (Amgen Inc., Thousand Oaks, Calif.). All cultures were incubated at 37° C. and 5% CO₂. Colonies were counted between day 10 and 12. In certain experiments, colony forming unit (CFU)-erythroid (CFU-E), CFU-monocyte (CFU-M), CFU-granulocyte/monocyte (CFU-GM) and CFU-granulocyte/erythroid/monocyte/megakaryocyte (CFU-GEMM) were enumerated separately based on their characteristic morphologies as described.

### Transplantation Assays

Radioprotection Assay

5×10⁴ fresh or cultured Lin− BM cells were transplanted into lethally irradiated CD45.2+ mice (1,000cGy). Animals were evaluated as described below in competitive repopulation assay.

Competitive Repopulation Assay

For competitive repopulation studies, CD45.2 recipient mice were irradiated with 950-1,100 cGy 3 to 12 hours prior to transplantation. 2×10⁵ BMMNC cells from CD45.2 mice were mixed with fresh or 3-week culture progeny of 10⁴ Lin− CD45.1 cells. For experiments using KLS population 200 d0 KLS or d5 progeny from 200 d0 KLS cell were competed against 2×10⁵ BMMNC. After 1-4 months PB and/or BM was obtained and stained with FITC anti-CD45.1 (A20) and PerCp-conjugated anti-CD45.2 (104). The cells were simultaneously stained with APC-conjugated anti-B220 antibody together with a mixture of PE-conjugated anti-Mac-1 (M1/70) and -Gr-1 (RB6-8C5) or anti-CD4 (GK1.5) and -CD8 (53-6.7) antibodies (BD Pharmingen). Four-color analysis was performed on a FACSCaliber or FACSCanto (BD Biosciences, San Jose, Calif.). A recipient mouse was considered multi-lineage repopulated if the percentage of donor cell-derived cells was >1% and donor cells contributed to all three hematopoietic lineages (myeloid, T lymphoid and B lymphoid cells) in PB and/or BM. Competitive repopulation assays were repeated 2 to 4 times with separate isolations of lineage negative or KLS BMMNC.

Secondary Transplantation

Animals with >5% donor cells in the BM were used as donors for secondary transplantation. 1×10⁶ total BM cells obtained from primary recipients were injected into 3-5 lethally irradiated CD45.2 secondary recipients.

### Western Blot

Cell pellets from stromal cells were resuspended in ice-cold RIPA buffer (Sigma) with protease inhibitor tablets. Samples were then centrifuged at 14,000 rpm at 4° C. for 10 minutes, and supernatant recovered. An equal volume of 2× sample buffer (Invitrogen, Carlsbad, Calif.) was added and samples were placed at 90° C. for 10 minutes. Samples were separated on 4-12% (SDS-PAGE) gels (Invitrogen). Gels were transferred to Immuno-Blot PVDF membrane (BioRad, Hercules, Calif.) for 2 hours. Membranes were blocked using 5% nonfat dry milk in PBS-T (pH 7.6, 0.1% Tween-20 in PBS for 1 hour at room temperature and then incubated overnight with specific primary antibodies at 4° C. Primary antibodies used were against Wnt5a (R&D Systems), β-catenin, activated 13-catenin (abc) (clone name Millipore; Upstate, Billerica, Mass.), and Disheveled-2 (Dvl-2) (clone name Santa Cruz, Santa Cruz, Calif.). Blots were then washed 3× for 5 minutes with PBS-T, and incubated with secondary antibodies in PBS-T according to the manufacturer's protocol. Bands were visualized using secondary horseradish peroxidase-conjugated Abs and chemiluminescence (Amersham, GE Healthcare Biosciences Piscataway, N.J.).

### Quantitative RT-PCR

Total RNA was harvested from the to be tested cells cells using the Qiagen RNeasy Kit (Qiagen, Hilden, Germany). Total RNA was Dnase treated. RNA was quantified by absorbance at A260 nm and 2 μg of total RNA used for cDNA synthesis using Taqman reverse transcription reagents. Q-RT-PCR was carried out using Taqman SYBR green universal mix PCR reaction buffer using an ABI PRISM 7700 (Perkin Elmer Applied Biosystems, Boston, Mass.).

Q-RT-PCR primers

| | | |
|---|---|---|
| Tfpi | F1 | GCCAGATTTCTGCTTCTTGG |
| | | (SEQ ID NO: 1) |
| | R1 | GGGAGTGGACTGGATTCTCA |
| | | (SEQ ID NO: 2) |
| Matn3 | F1 | GCATCTGGCATTGAGCTGTA |
| | | (SEQ ID NO: 3) |
| | R1 | GCACTGATCCAGAGCACAAA |
| | | (SEQ ID NO: 4) |
| Igfbp3 | F1 | CAACCTGCTCCAGGAAACAT |
| | | (SEQ ID NO: 5) |
| | R1 | AACTTTGTAGCGCTGGCTGT |
| | | (SEQ ID NO: 6) |
| Nxph1 | F1 | GGCCGTAGTAGCTCCTCCAT |
| | | (SEQ ID NO: 7) |
| | R1 | GCCTTGGCTGAGTCTTTGTT |
| | | (SEQ ID NO: 8) |
| Prg4 | F1 | GGCAAGTGCTGTGCAGATTA |
| | | (SEQ ID NO: 9) |
| | F2 | GAAGAGGAGGAGGAGGAGGA |
| | | (SEQ ID NO: 10) |
| Ctgf | F1 | CAAAGCAGCTGCAAATACCA |
| | | (SEQ ID NO: 11) |
| | R1 | GGCCAAATGTGTCTTCCAGT |
| | | (SEQ ID NO: 12) |
| Dlk-1 | F1 | TGTCAATGGAGTCTGCAAGG |
| | | (SEQ ID NO: 13) |
| | R1 | AGGGAGAACCATTGATCACG |
| | | (SEQ ID NO: 14) |

Quantitative RT-PCR primers for Wnt

| | | |
|---|---|---|
| mWnt1 | F1 | CTACTGGCACTGACCGCTCT |
| | R1 | GAATCCGTCAACAGGTTCGT |
| mWnt2 | F1 | GCAACACCCTGGACAGAGAT |
| | R1 | ACAACGCCAGCTGAAGAGAT |
| mWnt2b | F1 | ACTGGGGTGGCTGTAGTGAC |
| | R1 | ACCACAGCGGTTGTTGTGTA |
| mWnt3 | F1 | TTCCCATCTCTCCTTCCAGA |
| | R1 | CCCGGAAACATGACTTTATCA |
| mWnt3a | F1 | GCCTGGAAGTGAGGTGCTAC |
| | R1 | ACTCCTGTATGGCCACATCC |
| mWnt4 | F1 | CTGGAGAAGTGTGGCTGTGA |
| | R1 | GGACGTCCACAAAGGACTGT |
| mWnt5a | F1 | AATCCACGCTAAGGGTTCCT |
| | R1 | GAGCCAGACACTCCATGACA |
| mWnt5b | F1 | GCATCAGAGAGTGCCAACAC |
| | R1 | GCTCACTGCATACGTGAAGG |
| mWnt6 | F1 | CTTCGGGGATGAGAAGTCAA |
| | R1 | AGCCCATGGCACTTACACTC |
| mWnt7a | F1 | TCGGGAAGGAGCTCAAAGT |
| | R1 | GCCACAGTCGCTCAGGTT |
| mWnt7b | F1 | ATGCCCGTGAGATCAAAAAG |
| | R1 | GTGGTCCAGCAAGTTTTGGT |
| mWnt8a | F1 | AACGGTGGAATTGTCCTGAG |
| | R1 | GGTGACTGCGTACATGATGG |
| mWnt8b | F1 | GTGGACTTCGAAGCGCTAAC |
| | R1 | CAGTTGTCCATTTCGGGAGT |
| mWnt9a | F1 | GTACCAGTTCCGCTTTGAGC |
| | R1 | AGATGGCGTAGAGGAAAGCA |
| mWnt9b | F1 | ACAGCACCAAGTTCCTCAGC |
| | R1 | GCACTTGCAGGTTGTTCTCA |
| mWnt10a | F1 | CCGAGGTTTTCGAGAGAGTG |
| | R1 | AACCGCAAGCCTTCAGTTTA |
| mWnt10b | F1 | TTCTCTCGGGATTTCTTGGA |
| | R1 | CGCTTCAGGTTTTCCGTTAC |
| mWnt11 | F1 | TTCCGATGCTCCTATGAAGG |
| | R1 | CCCCATGGCATTTACACTTC |
| mWnt16 | F1 | CACCACAGGAAAAGAAAGCAG |
| | R1 | CAGGTTTTCACAGCACAGGA |
| mFzd1 | F1 | CACCTGGATAGGCATCTGGT |
| | R1 | GTAACAGCCGGACAGGAAAA |
| mFzd2 | F1 | CCGTCTCTGGATCCTCACAT |
| | R1 | TAGCAGCCGGACAGAAAGAT |
| mFzd3 | F1 | GAAGCAAAGCAGGGAGTGTC |
| | R1 | ATGCTGCCGTGAGGTAGTCT |
| mFzd4 | F1 | AACCTCGGCTACAACGTGAC |
| | R1 | TGGCACATAAACCGAACAAA |
| mFzd5 | F1 | CTGTGGTCTGTGCTGTGCTT |
| | F2 | TCCCAGTGACACACACAGGT |
| mFzd6 | F1 | CCTTACCAGGCAAATCCAAA |
| | R1 | TGTGCACGTCTTTTTGCTTC |
| mFzd7 | F1 | CCATCCTCTTCATGGTGCTT |
| | R1 | ATGGCCAAAATGGTGATTGT |
| mFzd8 | F1 | TCCGTTCAGTCATCAAGCAG |
| | R1 | CGGTTGTGCTGCTCATAGAA |
| mRor1 | F1 | GATTTATTGGCAACCGCACT |
| | R1 | ATGGCGAACTGAGAGCACTT |
| mRor2 | F1 | CACTGCCACTGGGGTTCTAT |
| | R1 | TAGTCCGGTTCCCAATGAAG |

### Single Cell Immunofluorescent Staining

CD34-KLS cells were sorted into 30 μl Stemspan (Stem Cell Technologies) on glass slides. After sorting slides were placed on ice for 1 hour and then supplemented with 50 ng/ml SCF, 100 ng/ml Tpo and 100 ng/ml Wnt5a, 100 ng/ml BMP-4, and/or 5 μg/ml Tsg (R&D Systems). Slides were incubated at 37° C. for 2 to 18 hours as indicated in figure legends. Following incubated 30 μl of 10% NBF was added for 10 minutes, and permeabalized for 10 minutes using 0.02% Triton-X100. Slides were blocked with 10% goat serum (Abcam, Cambridge, UK), and stained with primary antibodies overnight. The cells were washed with PBS, and stained with secondary antibody (Invitrogen) and Hoechst for 30 minutes. Protocol was adapted from Ema, et al. [6].

### Processing of RNA Samples and Microarray Analysis.

Total cellular RNA was isolated using the RNAeasy microkit (Qiagen, Valencia, Calif.) from UG26-1B6, AFT024 and EL08-1D2 stromal cells (in triplicate) that had been irradiated with 2,500cGy per manufacturer's instructions. 2 μg of RNA was then used for cDNA synthesis, which was performed using Superscript double stranded cDNA synthesis kit (Invitrogen, Carlsbad, Calif.) followed by labeling with the Enzo Bioarray HighYield RNA Transcript Labeling Kit (Enzo Life Sciences, Farmingdale, N.Y., United States) according to manufacturer's instructions. Samples were hybridized to Affymetrix mouse 430 2.0 chips (Affymetrix Inc), washed, and scanned at the University of Minnesota Affymetrix Microarray Core Facility as described in the Affymetrix GeneChip Expression Analysis Technical Manual.

CEL files were loaded into GeneData Expressionist Refiner (GeneData, San Francisco, Calif.) to assess overall quality and feature intensities for each chip were condensed into a single intensity value per gene using the Affymetrix Statistical Algorithm (MAS 5.0). Data was analyzed using GeneData's Expressionist and Microsoft Excel (Microsoft, Redmond, Wash., United States). Differential gene expression was defined by using a false discovery rate of <15% or <1% and a paired fold change was used to rank gene lists. Differentially expressed genes were classified according to their respective gene pathways and gene ontologies using Affymetrix NetAffx analysis tool (http://www.affymetrix.com) and Ingenuity database (www.ingenuity.com).

### Fzd4 KLS

Lineage depleted BM cells were isolated as described herein. Cells were stained with 1 μg/ml mouse anti-Frizzled 4 (R&D Systems, Minneapolis, Minn.) or 1 μg/ml IgG control antibody (Santa Cruz Biotechnology, Santa Cruz, Calif.) in 2% FCS (Stem Cell Technologies, Vancouver, BC, Canada) for 1 hour, followed by 30 minute staining with FITC conjugated anti-goat IgG (Santa Cruz Biotechnology). Afterwards, cells were stained with antibodies against Lineage positive cells, ScaI and cKit as described herein.

### Statistical Analysis

Data for studies determining cell number, cell phenotype, CFC number and transcript levels are shown as the mean of a minimum of three experiments±standard deviation. Differences in radio-protective assays and competitive repopulation were based on student's t-test. P-value of <0.05 was considered statistically significant.

### Example 1

### AGM Derived Stromal Cell Line Secretes Factor(s) Capable of Supporting Hematopoietic Stem Cells

### Introduction

Hematopoietic stem cells (HSC) are responsible for maintaining hematopoiesis throughout adult life. HSC fate decisions are regulated by both extrinsic and intrinsic signals. In vivo, extrinsic signals are emanated by the microenvironment where the cell resides. These signals are both direct cell-cell interaction based, wherein receptors on HSC bind to ligands on cells within their microenvironment (including among others osteoblasts, endothelial cells, and marrow stroma cells), cell-extracellular matrix (ECM) based (fibronectin, proteoglycans, among others), as well as, due to soluble factors secreted by cells residing in the microenvironment or present at a more distant location (e.g. erythropoietin secreted chiefly by cells in the kidney). The microenvironment wherein HSC reside differs throughout ontogeny, perhaps because distinct signals are needed for the proper development, expansion and lineage commitment of HSC [7]

Hematopoiesis is first seen in the yolk sac around E7.5 during mouse development, giving rise to primitive erythroid cells and macrophages, and as has recently been demonstrated also long term repopulating HSC that persist into adulthood [7, 8]. A second location where HSC are being generated is the aorta-gonad-mesonephros (AGM) region around E9.5 in mouse, where the hemogenic endothelium differentiates into HSC. Around E12.5, HSC populate the fetal liver (FL) where HSC expand and generate committed progenitors for all myeloid and lymphoid lineages. At birth, HSC migrate a final time from the FL to the bone marrow (BM) where hematopoiesis is maintained throughout adult life [9]. The different hematopoietic microenvironments are thought to be responsible for regulating initiation, expansion, and maintenance of hematopoiesis.

The microenvironment wherein HSC reside plays a key role in inducing either differentiation or self-renewal of HSC. For instance, interaction between osteoblasts and HSC in the BM has recently been shown to be a factor for in vivo self-renewal of HSC, as a result of, amongst others Notch/Jagged interactions [10, 11]. Dexter et al were the first to demonstrate that when whole BM is plated in culture in the presence of serum, a feeder of attached cells consisting of a mixture of macrophages, fibroblasts, large reticular cells and some endothelial cells (all together named marrow stroma) becomes established [12]. Underneath and between the adherent stromal cells, hematopoietic cell colonies can be found, some of which contain cells that can repopulate the hematopoietic system, even after several weeks of in vitro culture. Subsequently many investigators have isolated cell lines from different regions of the developing embryo and the adult BM, hypothesizing that characterization of such feeders might aid in the identification of factors that maintenance and expansion versus lineage commitment of HSC [1, 2, 12]. In so doing, it has become clear that some lines derived from postnatal BM support primitive progenitors, and others more committed progenitors in vitro [3, 12-15]. Stromal cell lines have also been derived from the FL. For instance, the AFT024 cell line generated from the FL of E14.5 mouse supports mouse long-term repopulating HSC (LTR-HSC) and human cells that engraft in non-obese diabetic-severe combined immunodeficiency (NOD-SCID) mice, whereas other lines generated from FL from similar aged fetuses, only support committee progenitors or do not support hematopoiesis at all [2, 16]. Oostendorp et al. generated a series of stromal cell lines from different subregions of the AGM of E10.5 mouse embryos, specifically the urogenital ridge (UG), as well as E11 embryonic liver (EL) [1, 17]. Two of these cell lines, EL08-1D2 and UG26-1B6, support murine and human long-term culture initiating cells (LTC-IC) and mouse LTR-HSC and human NOD-SCID repopulating cells [1, 17-19]. As only some cell lines generated from the different hematopoietic microenvironments at the same stage of development support repopulating HSC in vitro, comparing the cell surface antigens, and secreted ECM components and soluble factors of supportive and non-supportive feeders might shed light on microenvironment components responsible for HSC maintenance and/or proliferation [3].

To elucidate factors produced by the AGM and EL/FL feeders that support HSC maintenance in vitro, the maintenance of competitive repopulating HSC from adult mice cultured in direct contact with or transwells above UG26-1B6, EL08-1D2 and AFT024 feeders was evaluated.

### Results

The Urogenital Ridge and Liver-Derived Feeders Maintain Committed Progenitors for 3 Weeks in Vitro, Both in Contact and Non-Contact Cultures.

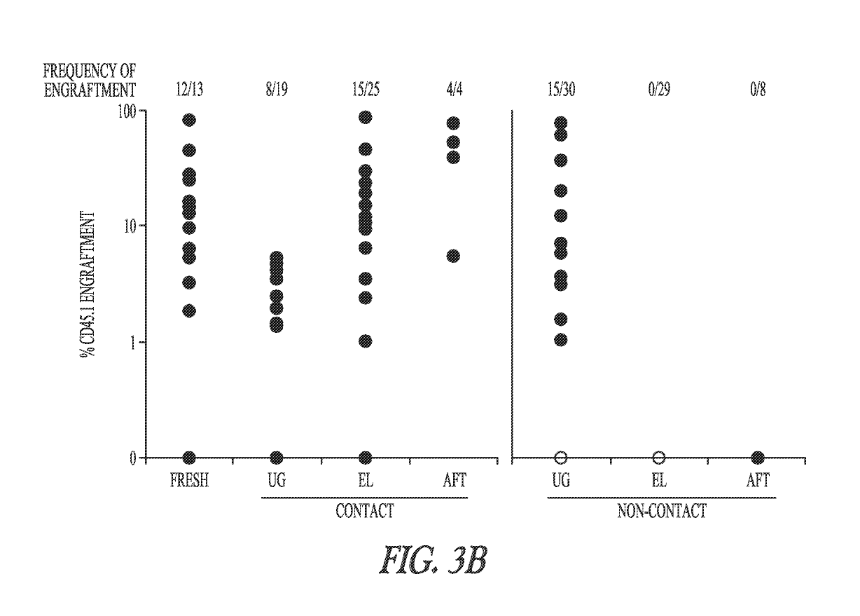

Murine Lin− BM cells were plated in transwells above or in direct contact with the UG26-1B6, EL08-1D2 and AFT024 feeders. Three weeks later progeny was evaluated for total cell expansion and presence of colony forming cells (CFC). The total cell number in contact cultures using any of the three feeders was identical at three weeks, whereas significantly fewer cells were present in AFT024 non-contact cultures than in UG26-1B6 and EL08-1D2 non-contact cultures (FIG. 3A). FACS analysis at 3 weeks demonstrated that the majority of progeny cells were Gr-1 and Mac-1 double positive, irrespective of the culture condition.

The number of CFC recovered after 3 weeks from contact cultures was similar for the three feeders (n=3). However, fewer CFC were supported in AFT024 non-contact cultures than when cells were plated in transwells above the other two feeders (n=3) (FIG. 3B), suggesting that in contrast to UG26-1B6 and EL08-1D2 cells, soluble factors produced by AFT024 are insufficient to support HPC.

Long-Term Repopulating HSCs are Maintained when Cultured in UG26-1B6, but not EL08-1D2 or AFT024 non-contact cultures.

It was next tested whether soluble factors from the different feeders can support HSC. First the ability of progeny of Lin− BM cells from CD45.1 mice cultured for 3 weeks in contact with or in transwells above UG26-1B6 and EL08-1D2 feeders to radioprotect lethally irradiated (1,000 cGy) congenic CD45.2 animals was tested. 80% of animals transplanted with CD45.1⁺ Lin− BM cultured in contact with EL08-1D2 were engrafted with donor cells (n=5), whereas none of the animals transplanted with progeny of Lin− BM cells from non-contact EL08-1D2 cultures engrafted. In contrast 40% (n=5) and 100% (n=5) of animals receiving progeny of Lin− BM cells cultured in UG26-1B6-non-contact and -contact cultures, respectively, engrafted with CD45.1+ donor cells (Table 1). Animals that were not repopulated did not survive three months after transplant.

TABLE 1
In a radio protective assay Lin⁻BM cells cultured for 3 weeks with
UG26-1B6 in non-contact culture contain repopulating stem cells.

| | Number of mice repopulated vs. | |
|---|---|---|
| | mice transplanted | % Repopulated mice |
| Fresh | 2/3 | 67% |
| UG26-1B6 | 5/5 | 100% |
| contact | | |
| UG26-1B6 non- | 2/5 | 40% |
| contact | | |
| EL08-1D2 | 4/5 | 80% |
| contact | | |
| EL08-1D2 non- | 0/5 | 0% |
| contact | | |

5 × 10⁴ Lin− CD45.1⁺ BM cells were cultured in contact with or transwells above confluent irradiated feeders. After 3 weeks, progeny from the 5 × 10⁴ Lin− CD45.1⁺ BM cells were injected IV into C57BL/6J (CD45.2⁺) mice. PB and BM were analyzed by FACS after 4½ months.
Numbers represent animals in which donor derived repopulation was seen after 4½ months in the PB.

One million BM cells collected from two primary recipients that received Lin− BM cells from UG26-1B6 non-contact or contact cultures were grafted in lethally irradiated secondary animals. All secondary recipients showed multi-lineage repopulation by CD45.1+ cells demonstrating that soluble factors secreted by UG26-1B6 feeders can support LTR-HSC.

To further substantiate the HSC supportive ability of secreted factors from UG26-1B6 feeders, competitive repopulation assays were performed [20-22]. 10,000 Lin− BM cells from CD45.1⁺ mice were cultured in contact with or in transwells above UG26-1B6, EL08-1D2 and AFT024 feeders, and progeny competed with 200,000 BMMNC from CD45.2+ mice in lethally irradiated CD45.2+ mice. Repopulation from CD45.1+ donor cells was defined as presence of >1% CD45.1+ cells in blood and/or BM, 3-4 months post transplantation, with contribution to the Gr-1/Mac-1+ myeloid, B220+ B- and CD4/CD8+ T-lymphoid lineages.

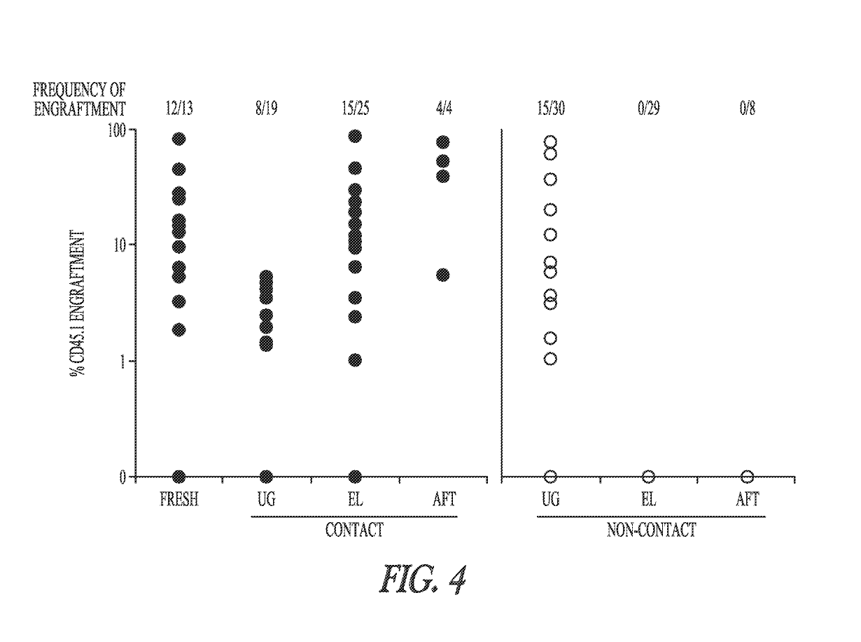

CD45.1+ hematopoiesis was seen in 12/13 mice transplanted with fresh CD45.1+ Lin− cells, (19±22% CD45.1 cells) (FIG. 4). Fifteen of 25 recipient mice showed chimerism when Lin-progeny from 3 week EL08-1D2-contact-cultures was competed with BMMNC from CD45.2+ mice (15±21% CD45.1+ cells). Transplantation of week-3 progeny from EL08-1D2 non-contact cultured cells did not yield detectable CD45.1+ cell repopulation (0/29 mice, p<0.001 compared with EL08-1D2 contact progeny), demonstrating again that EL08-1D2 cells support competitive repopulating HSC in contact but not in non-contact culture. Four of 4 mice grafted with progeny from AFT024-contact cultures (41±30% CD45.1+ cells), but 0/8 from AFT024 non-contact cultures had CD45.1+ hematopoiesis 4 months post transplantation. In contrast, UG26-1B6 feeders could maintain competitive repopulating HSC in both contact and in non-contact cultures: 8/19 mice transplanted with culture progeny of UG26-1B6 contact cultures met criteria for competitive repopulation by CD45.1+ cells (2±1.4% CD45.1+ cells), whereas, 15/30 mice transplanted with culture progeny of UG26-1B6 non-contact cultures showed multilineage repopulation with CD45.1+ cells (10±20% CD45.1+ cells) (FIG. 4).

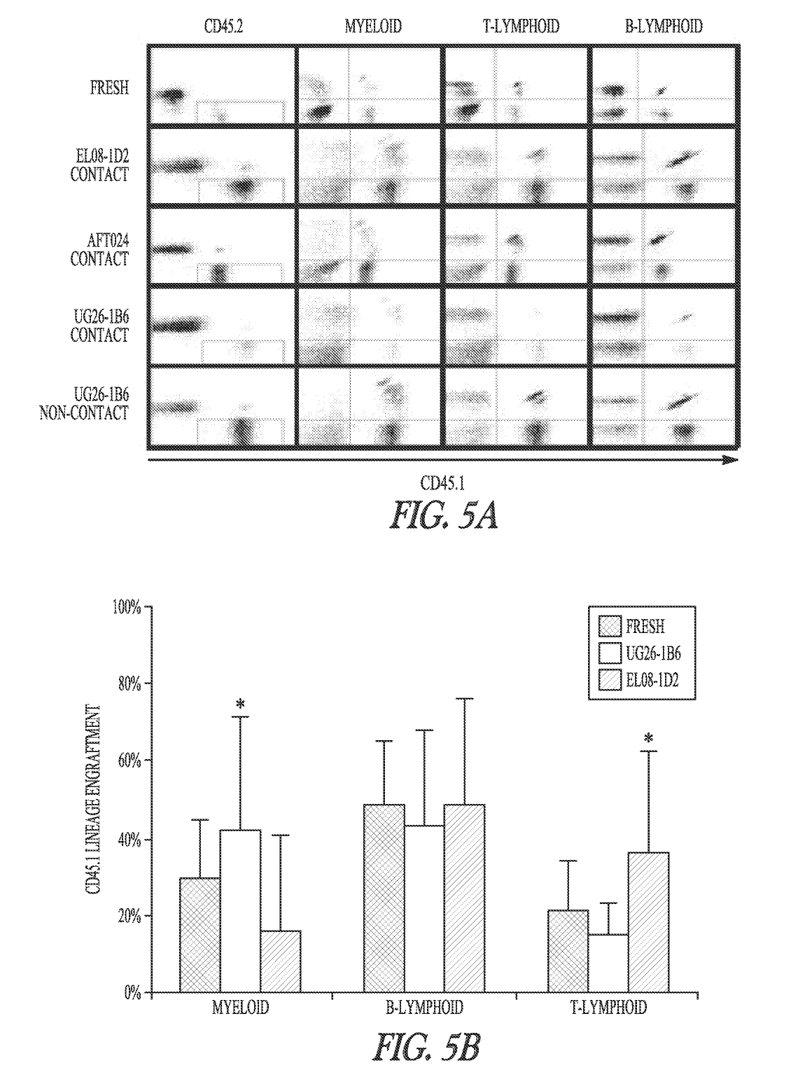

At first sight, the level of chimerism from cells cultured in contact with EL08-1D2 feeders was higher than the level of chimerism in animals grafted with cells maintained in UG26-1B6 non-contact or contact cultures. As defined above, all engrafted animals had contribution to the myeloid, B- and T-lymphoid lineages. FIG. 5 illustrates the multilineage potential of donor-derived cells from one representative individual mouse in each of the LTR-HSC supporting conditions. Multilineage engraftment in animals that received progeny of EL08-1D2-contact cultures was skewed towards T lymphocytes with a reduction in myeloid engraftment compared to uncultured BM cells and cells cultured in contact with UG26-1B6 (p-value<0.05) (FIG. 5). Of the 10/25 mice that did not meet the requirements for donor derived engraftment in the EL08-1D2 contact culture group, three mice were repopulated with CD45.1+ cells, but only in the lymphoid compartment, and one mouse showed only myeloid engraftment of CD45.1+ cells.

Secondary transplants were also performed to demonstrate that LTR-HSC had persisted in the different culture systems. One million BM cells from 2 animals with >5% CD45.1+ chimerism following transplantation of progeny from each of EL08-1D2 contact, AFT024-contact, and UG26-1B6-non-contact cultured Lin− BM cells 4 months earlier, was transferred to secondary recipients. Table 2 summarizes the chimerism of the 6 primary recipients from which the BM was used for secondary transplants. Three and four of four of the secondary recipients of BM cells from animals grafted with EL08-1D2-contact #1 or #2 progeny, respectively, had multilineage CD45.1+ hematopoiesis, but engraftment was skewed towards the B- and T-lymphoid lineage, respectively. Four and zero of four of the secondary recipients of BM cells harvested from animals grafted with AFT024-contact #1 or #2 progeny showed multilineage CD45.1+ hematopoiesis, but—as for EL08-1D2-contact secondary recipients—also skewed towards the lymphoid lineages. Both groups of mice that received cells from primary recipients repopulated with UG26-1B6 non-contact cultured cells repopulated with CD45.1+ cells, which was multilineage in 6/8 secondary recipients.

TABLE 2

| | in primary | Percent of donor derived cells in secondary recipients | | | |
|---|---|---|---|---|---|
| (n = 4) | recipient PB | Total | Myeloid | B Lymphoid | T Lymphoid |
| EL08-1D2 | 31.8% | 2.5 ± 1.7 | 11.6 ± 7.7 | 71.2 ± 19.6 | 17.2 ± 15.2 |
| contact#1 | | | | | |
| EL08-1D3 | 86.7% | 43.7 ± 7.1 | 10.3 ± 4.2 | 13.5 ± 15.5 | 79.3 ± 12.4 |
| contact#2 | | | | | |
| UG26-1B6 | 7.1% | 68.9 ± 6.3 | 53.6 ± 15.5 | 5.7 ± 1.4 | 40.7 ± 14.3 |
| non-contact#1 | | | | | |
| UG26-1B6 | 5.7% | 7.8 ± 14.9 | 64.9 ± 10.6 | 21.6 ± 13.5 | 13.5 ± 15.7 |
| non-contact#2 | | | | | |
| AFT024 | 85.7% | 29.3 ± 6.6 | 6.7 ± 2.8 | 32.3 ± 9.6 | 67.4 ± 11.8 |
| contact#1 | | | | | |
| AFT024 | 49.7% | 9.5 ± 8.2 | 20.8 ± 31.9 | 0.0 ± 0.0 | 79.2 ± 31.9 |
| contact#2 | | | | | |

±stdev
Culture of Lin− BM cell in UG26-1B6 non-contact cultures preserves LTR-HSC. 1 × 10⁴ Lin− CD45.1⁺ BM cells were cultured in contact with or transwells above confluent irradiated feeders. After 3 weeks, progeny from the 1 × 10⁴ Lin− CD45.1⁺ BM cells were transplanted with 2 × 10⁵ fresh CD45.2⁺ BMMNC IV into C57BL/6J (CD45.2⁺) mice.
10⁶ TBM cells from two separate primary recipients were injected into groups of four CD45.2⁺ secondary recipients for each primary. The two separate primary recipients that were used for secondary transplantations are labeled #1 and #2.
Peripheral blood was collected 3 months after transplantation, and analyzed by FACS for CD45.1⁺ derived cells.
The total contribution of donor CD45.1⁺ cells or donor myeloid, B- or T-lymphoid cells was determined as described in the Materials and Methods section.

UG26-1B6 Secretes a Factor or Factors Capable of Supporting Hsc in Culture.

To determine whether lack of HSC maintenance in EL08-1D2 non-contact cultures is due to secretion of an inhibitor by the cells, or whether EL08-1D2 cells fail to secrete one or more soluble factors capable of maintaining HSC, UG26-1B6 and EL08-1D2 cells were mixed at different ratios. If addition of 25% UG26-1B6 cells to 75% EL08-1D2 cells would lead to maintenance of HSC, then UG26-1B6 cells must secrete a factor that supports HSC. If, by contrast, combining 75% UG26-1B6 cells with 25% EL08-1D2 cells does not lead to HSC support, a factor produced by EL08-1D2 may inhibit HSC maintenance. To distinguish between the two cell lines, UG26-1B6 cells were transduced with a retroviral vector encoding the enhanced green fluorescent protein (eGFP) and the GFP+ cells sorted by FACS. The relative proportion of UG26-1B6 and EL08-1D2 present in culture after plating the different ratios was verified by FACS and fluorescence microscopy.

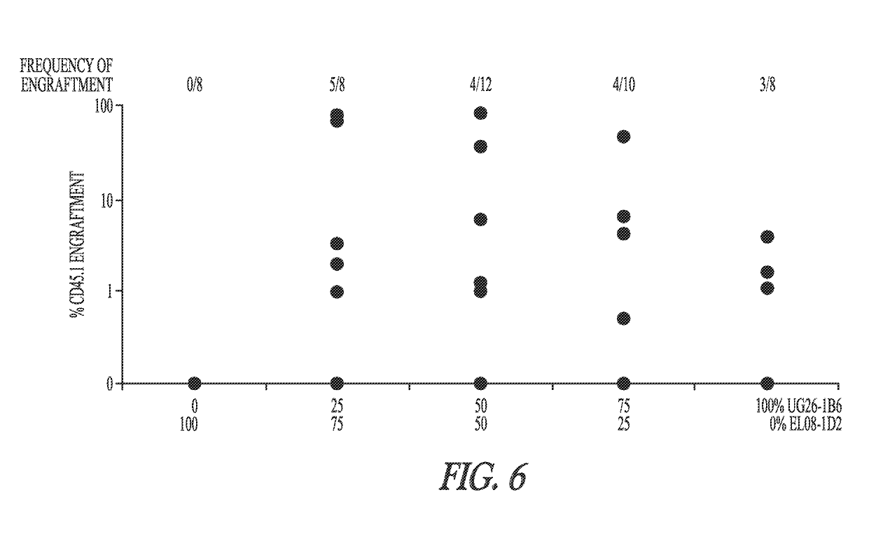

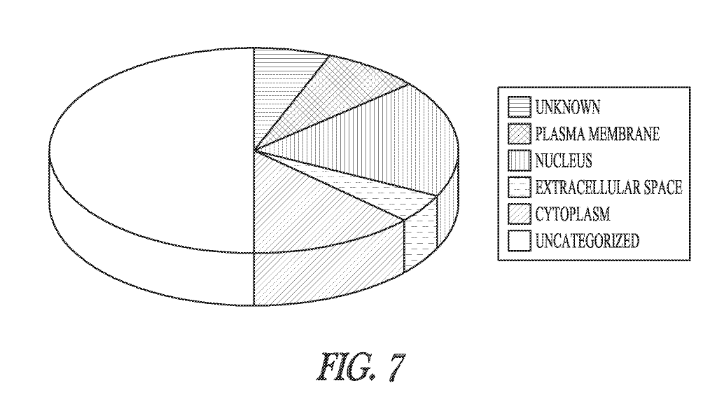

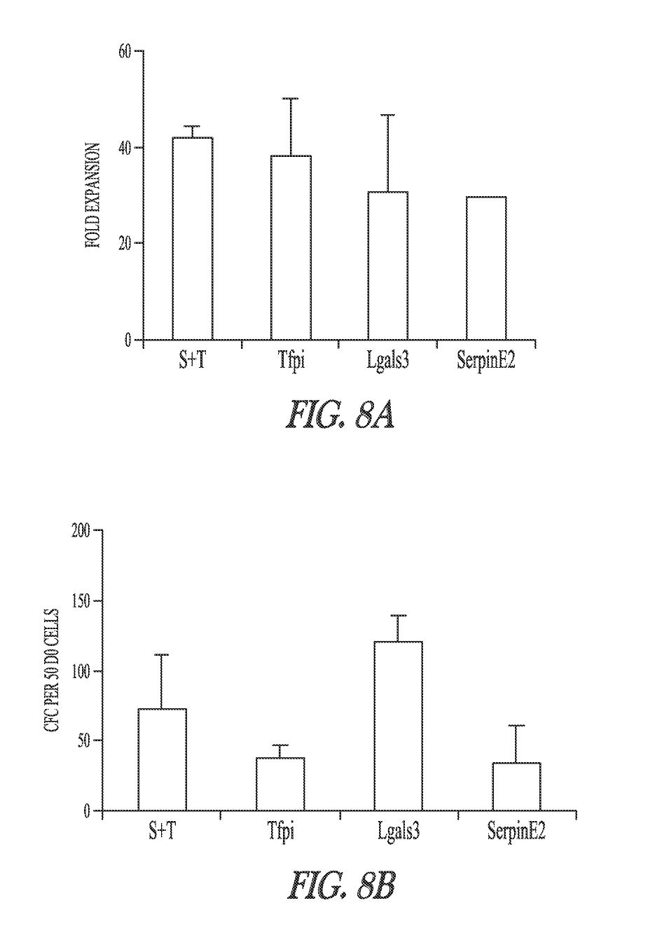

Total cell expansion and recovery of CFC at 3 weeks was similar between cells maintained in any of the non-contact cultures above mixed stromal cell lines. Lin− CD45.1+ BM cell progeny from the different cultures were transplanted in a competitive repopulation assays with CD45.2+ cells. Addition of as little as 25% UG26-1B6 cells to the EL08-1D2 stromal cell cultures resulted in the maintenance of HSC in non-contact culture. 50% of transplanted mice demonstrated multi-lineage engraftment with >1% donor derived cells. Similar multilineage engraftment was seen in mice receiving progeny of cultures containing 100%, 75%, 50%, or 25% UG26-1B6(FIG. 6) indicating that EL08-1D2 fails to secrete a factor needed for HSC support in non-contact cultures. Lymphoid skewing was not seen in recipients of cells cultured with mixed stromal cell lines. These studies demonstrate that UG26-1B6 secretes a factor or multiple factors needed for HSC maintenance in non-contact culture, not produced by EL08-1D2 cells.

### Discussion

The nature of signals that govern HSC self-renewal, differentiation and proliferation are still not well understood. Throughout development, HSC reside in multiple different sites [9, 23]. It is therefore thought that characterization of the microenvironment of these developmentally diverse sites may yield important insights in factors that regulate HSC behavior.

Herein it is demonstrated that feeders generated from E10.5-AGM, E10.5-EL and E14.5-FL support HSC in vitro, but that contact with UG26-1B6 cells is not required to maintain HSC, even though there was no HSC expansion. These results suggest that one or more soluble factors are secreted by the UG26-1B6 cell line that can support HSC in vitro, whereas such factor(s) are not secreted by the EL08-1D2 or AFT-024 feeders. Although a number of studies have characterized which established hematopoietic cytokines and growth factors are secreted by different feeders, no specific pattern of expression has been associated with the ability of one but not other feeders to support primitive hematopoietic progenitors. A number of recent studies have suggested that morphogens operative during early embryonic development may play a role in HSC self-renewal and differentiation. Hedgehog (HH) proteins are involved in embryonic as well as post-natal hematopoiesis. For instance, Indian HH is involved in the induction of primitive hematopoiesis in mouse embryos through up-regulation of bone morphogenetic protein (BMP)-4 expression [24, 25]. More recently, a role for HH proteins in adult hematopoiesis has been recognized, as addition of exogenous sonic HH to stroma-free cultures of human CD34+38-Lin− cells, resulted in expansion of SCID-repopulating cells (SRC)[26]. Like HH proteins, BMPs are evolutionarily conserved. Although it has long been known that BMPs play a pivotal role in ventral mesoderm specification and induction of primitive hematopoiesis during embryonic development, recent evidence has been obtained that BMPs may also play a role in later stages of hematopoietic development. For instance addition of BMP-4 to cultures of primitive CD34+38−Lin− cells, results in increased preservation of SRC [26, 27]. A third family of proteins known to play a role in development, including hematopoiesis, is the Wnt family. Components of the Wnt pathway promote proliferation of stem/progenitor cells of skin [28], gut [29], brain [30] as well as embryonic stem cells [31]. A number of studies have shown that several Wnt members, including Wnt3, Wnt-5a and Wnt-10b affect self-renewal of human or mouse HPC and HSC [32-35]. Wnt-5a and Wnt-10b are expressed in the yolk sac and embryonic liver[32], and Wnt-5a is expressed between E10 and E11 in the AGM region [36].

In the examples provided herein, the molecular differences between the three stromal feeders will be defined, which aids in the identification of factors that regulate HSC self-renewal. Specifically, the transcriptome of the different feeders will be compared, differentially expressed transcripts will be identified and tested to determine whether these may play a role in the HSC supportive nature of UG26-1B6 non-contact cultures.

A second finding was that secondary transplantation of progeny of EL08-1D2 or AFT-024 contact cultures yielded mainly lymphoid repopulation. One possible explanation is that the number of LTR-HSC maintained in the EL08-1D2 contact cultures is limited, and mainly lymphoid progenitors are passed to the secondary recipient. Here too, evaluation of the expressed gene profile of EL08-1D2 cells will shed light on which factors expressed by this feeder may be responsible for maintenance of lymphoid HPC and to a lesser extent multilineage LTR-HSC.

### Example 2

### Gene Expression Profiling of HSC Supportive Stromal Cell Lines

### Introduction

The HSC niche consists of “stromal” cells (including osteoblasts, endothelial cells, myofibroblasts, and macrophages) and extracellular matrix (ECM) with which HSC can interact, as well as secreted factors that affect HSC, all of which regulate cell fate decisions. Although many cytokines and growth factors have been isolated that affect HSC, none have been identified that can support HSC maintenance long-term (more than 1 week) in vitro. Stromal cell lines have been isolated from different HSC supportive environments during embryonic development and have been used to mimic the microenvironment of the HSC. We (and others) have found that some of these feeders support HSC maintenance, albeit no real expansion has been achieved in vitro. Initially it was believed that stromal cell lines could only support primitive hematopoietic cells that were plated in contact with the stromal cells, as hematopoietic cells are found in so called cobblestone areas, wherein the more primitive cells are actually underneath the stromal cells [37]. However, more recent studies have demonstrated that HSC can also be maintained using culture systems where direct contact between the HSC and stromal cells themselves is prevented by plating cells in a transwell placed above the stromal cells, which still allows for secreted factors to reach the HSC, but prevents direct HSC-stromal cell interactions [38-41]. Such a culture system has been named a “stroma-non-contact” culture. That primitive HPC/HSC can be maintained when cultured in transwells above stromal cells was first shown for mixed BM stromal cells [41] and subsequently for a variety of clonal stromal cells from different hematopoietic microenvironments [1, 3, 42]. Because some cell lines support HSC significantly better than others when hematopoietic cells are cultured in transwells above the feeders, comparing the secretome of the different feeders will yield insights in secreted factors that govern HSC maintenance.

A cell line derived from urogenital ridge of E10.5 mice (UG26-1B6 cells) was demonstrated to support maintenance of murine LTR-HSC when plated separate from the feeder by a 0.4 mm pore transwell, suggesting that UG26-1B6 cells secrete factors for HSC maintenance. By contrast murine LTR-HSC could not be maintained in transwells above another E10.5 derived feeder, namely the embryonic liver line, EL08-1D2. Murine LTR-HSC could be maintained when cultured in direct contact with the two feeders.

As these studies suggest that UG26-1B6 cells may secrete one or more factors that can support murine HSC in vitro, a transcriptome analysis of UG26-1B6 and EL08-1D2 co-cultured for 7 days with Lin− BM cells in transwells above the feeder to identify candidate factors was performed.

### Results

Identification of Secreted Factors Expressed More Highly in Irradiated UG26-1B6 than AFT024 and EL08-1D2 cells co-cultured with Lin− BM cells

A number of studies have demonstrated that there is cross talk between hematopoietic cells and stromal cells both in vivo and in vitro, and that such crosstalk also occurs when hematopoietic cells are cultured separated by a transwell from the stromal cells [1, 18, 43, 44]. For the transcriptome analysis, we used RNA isolated from UG26-1B6 and EL08-1D2 cells after they had been co-cultured with Lin− BM cells plated in transwells above the feeders for 7 days. The stromal cell lines were cultured to confluence and irradiated at 2,500cGy. After 24 hours, 10⁴ freshly isolated Lin− BM cells were plated in transwells above the feeders. After 7 days, the transwells containing the Lin− BM cells were removed and total RNA was extracted from the stromal cells. Labeled cRNA was generated and hybridized to the mouse 430 2.0 Affymetrix arrays containing >45,000 probes representing 34,000 genes. All studies were done in triplicate using RNA harvested from feeder cells cultured for different passages. The triplicate samples were normalized using a MAS 5.0 value of 140 for the fluorescence intensity of individual chips in order to compare the chips. The genes were then screened for absent or present call, if neither UG26-1B6 nor EL08-1D2 expressed a probe set, the probe was removed from the list. From there the individual groups of chips were analyzed and considered present if 2 out of the 3 chips expressed the probe set. Using these criteria, 22,451 probe sets were identified in at least one of the groups, UG26-1B6 or E108-1D2. The average of the replicates was used to determine the genes that were at least two-fold differentially expressed between the stromal cell lines, and the significance was determined using a false-discovery rate (FDR) of less than 1%.

1,834 genes were identified that were differentially expressed between UG26-1B6 and EL08-1D2 cells. Using the Ingenuity database (www.ingenuity.com) the genes were categorized by their cellular component, and 18 genes were classified as expressed in the extracellular space (FIG. 7). None (except Wnt5A, Dlk1 and Igfbp3) of the differentially expressed genes encode for cytokines or growth factors previously identified to influence HSC behavior. Indeed transcripts for stem cell factor (SCF), thrombopoietin (Tpo), Flt-3-L, along with non-classical hematopoietic morphogens and cytokines, such as Igf-2, Wn3a, Angiopoietin like proteins were not differentially expressed [5, 33, 34, 45-47]. Table 3 includes the 18 differentially expressed genes found higher expressed in UG26-1B6 compared to EL08-1D2. RT-qPCR was used to confirm the array data. These studies confirmed that Dlk-1, Tfpi, Nxphl, and Igfbp3 were more highly expressed in UG26-1B6 than EL08-1D2 cells, whereas transcript levels for Prg4 and Ctgf were similar in the two cell lines.

TABLE 3
Genes higher expressed by UG26-1B6 found in the extracellular space.

| | | Fold |
|---|---|---|
| Gene Symbol | Gene | Change |
| DLK1 | delta-like 1 homolog | 100 |
| TFPI | tissue factor pathway inhibitor | 20 |
| (1451791_at) | | |
| NXPH1 | neurexophilin 1 | 16 |
| PRG4 | proteoglycan 4 | 10 |
| CTGF | connective tissue growth factor | 9 |
| IGFBP3 | insulin-like growth factor binding protein 3 | 8 |
| DEFCR3 | defensin related cryptdin 3 | 5 |
| WNT5A | wingless-type MMTV integration site | 5 |
| (1436791_at) | family, member 5A | |
| SERPINE2 | serpin peptidase inhibitor, clade E, member 2 | 5 |
| COL18A1 | collagen, type XVIII, alpha 1 | 4 |
| EDN1 | endothelin 1 | 3 |
| (1451924_a_at) | | |
| BGLAP | bone gamma-carboxyglutamate protein | 3 |
| (1449880_s_at) | | |
| COL8A1 | collagen, type VIII, alpha 1 | 3 |
| PRG1 | proteoglycan 1, secretory granule | 3 |
| (1417426_at) | | |
| INHBA | inhibin, beta A | 2 |
| LTBP2 | latent transforming growth factor beta | 2 |
| | binding protein 2 | |
| MAC30 | hypothetical protein MAC30 | 2 |
| LGALS3 | lectin, galactoside-binding, soluble, 3 | 2 |

The sequences of the genes described herein are available to one of skill in the art. For example, the cDNA sequence of human neurexophilin 1 (NXPH1; Accession No. NM_{—}152745 (2931 by mRNA) is (SEQ ID NO:15):

| | |
|---|---|
| 1 | aagtaattgt ccgtgtcagg aaggtaggcg tgccaagccg cggctctgcg gagaaaccac |
| 61 | gaccaccgcg gccgccggaa acccaaagcg ctccagagcg tccccgggtg gccgggcagc |
| 121 | accagggaca gcgcccggga ctccactggg gaccggctcc tgggcttccc agcgtcgcgg |
| 181 | gtagaggtac agctgctccg tgtgccgcag gctccagatt ctcgccaccc cacccctccc |
| 241 | tcagaaactc ggactgctct cgtctgccgt gtggttctct tttcttccga aaggccagtg |
| 301 | tcttatctct ccacttcaag tccagaggac ttgctcagtc tcctcccctt aagtcatttc |
| 361 | caccatcctc aggcagctgt gggaagccga gagtcctgga ctgttcgtcc gggtgccagc |
| 421 | gctggcagtc ccagtccgtc cggtgcagca gcccggcgca ttcccctctc tccctccctc |
| 481 | ttgctctccc tccctttctg tcttcctctc tttcctcctc tactgctccc tccctctctt |
| 541 | gcctcttaag tttcctgcac cgtgaatcca actgtgccaa gccttggctc ccgcgaacca |
| 601 | atcctgagcg cgacccgggc actgggacgg cgactccgcc aaagctggac gaggcagccg |
| 661 | gacccgtctg cgctcgagca tggagacgga gcgcctggga gggcacgtcc ggggcgctgg |
| 721 | agacgccagg cccgagtagc ttctccatgg agcctgccca gagcggtccc ttctcgcagg |
| 781 | attcgcccca agtcctgtgc ggctgctgag agcgctcctt gctctgtaaa gtggatgtca |
| 841 | ggtggatcta tgtttctgaa ggaacaaaga ctcaaagaag gcaccgccaa ggaagtttga |
| 901 | gacgcgggag aatgcaggct gcgtgctggt acgtgctttt cctcctgcag cccaccgtct |
| 961 | acttggtcac atgtgccaat ttaacgaacg gtggaaagtc agaacttctg aaatcaggaa |
| 1021 | gcagcaaatc cacactaaag cacatatgga cagaaagcag caaagacttg tctatcagcc |
| 1081 | gactcctgtc acagactttt cgtggcaaag agaatgatac agatttggac ctgagatatg |
| 1141 | acaccccaga accttattct gagcaagacc tctgggactg gctgaggaac tccacagacc |
| 1201 | ttcaagagcc tcggcccagg gccaagagaa ggcccattgt taaaacgggc aagtttaaga |
| 1261 | aaatgtttgg atggggcgat tttcattcca acatcaaaac agtgaagctg aacctgttga |
| 1321 | taactgggaa aattgtagat catggcaatg ggacatttag tgtttatttc aggcataatt |
| 1381 | caactggtca agggaatgta tctgtcagct tggtaccccc tacaaaaatc gtggaatttg |
| 1441 | acttggcaca acaaaccgtg attgatgcca aagattccaa gtcttttaat tgtcgcattg |
| 1501 | aatatgaaaa ggttgacaag gctaccaaga acacactctg caactatgac ccttcaaaaa |
| 1561 | cctgttacca ggagcaaacc caaagtcatg tatcctggct ctgctccaag ccctttaagg |
| 1621 | tgatctgtat ttacatttcc ttttatagta cagattataa actggtacag aaagtgtgcc |
| 1681 | ctgactacaa ctaccacagt gacacacctt actttccctc gggatgaagg tgaacatggg |
| 1741 | ggtgagactg aagcctgagg aattaaaggt catatgacag ggctgttacc tcaaagaaga |
| 1801 | aggtcacatc tgttgcctgg aatgtgtcta cactgctgct cttgtcaact ggctgcaaaa |
| 1861 | tacactagtg gaaaacactc tgatgtaatt tctgcccagt cagcttcatc cctcagtata |
| 1921 | attgtaaatc atcacagatt ttgaattcac acctgaagac atgctctcac atatagaggt |
| 1981 | acacaaacac accgtcatgc acatttcagc ttgcgtctat catgattcct gttgagaggg |
| 2041 | ctttcattgt ctgactcata atggttcagg atcaactatc atcaaacgga aggattaact |
| 2101 | agacagagaa tgtttctaac agttgctgtt atggaaatct cttttaaagt cttgagtaca |
| 2161 | tgctaatcaa taatctccac tcatgcattc ctactgcttg gagtagctgt actggtaaat |
| 2221 | actactgtag gagtatctgc ttgttaaaat ggaaaaatgt gtctttagag ctcagtattc |
| 2281 | tttattttac aaacacaaca aaatgtagta acttttttcc agcatacagt aggcacattc |
| 2341 | aaagtggtcc aagatggctc ttttttcttt gaaaggggcc tgttctcagt aaagatgagc |
| 2401 | aaacatttgg aatttacatg tgggcagaca ttgggataac aactttcatc accaatcatt |
| 2461 | ggacttttgt gaagttgaca ccagctaagg ctgcttaaaa taagttctga tcattatata |
| 2521 | agaagggaaa tgcctggcag acaccatgta agttataagt gtctgtctta tctttactac |
| 2581 | acatattgta acaaattcaa tatcctagtc ttcatttgta tgaatggttt gtattgtaca |
| 2641 | tagtttaacc aagtgttatt tgagctgctt attaatatta acttgtactt gtctctctgc |
| 2701 | ttgttattgg ttaagaaaaa aggatatgag gaattcattt tatcaatgta gctgtgaagg |
| 2761 | ccattaaaaa gacaaactta atgtacagag catttattca gatcaagtat tgttgaaagc |
| 2821 | tatacatata caacattaca gtctgtctgt atttagatat tttatttctg gaaaaaatga |
| 2881 | aatgtacata aaaataaaac acttaaagtt gagtttcaat aaaaaaaaaa a |

The cDNA sequence of human endothelin 1 (EDN1; Accession No. NM_{—}001955 (2117 by mRNA) is (SEQ ID NO:16):

| | |
|---|---|
| 1 | ggagctgttt acccccactc taataggggt tcaatataaa aagccggcag agagctgtcc |
| 61 | aagtcagacg cgcctctgca tctgcgccag gcgaacgggt cctgcgcctc ctgcagtccc |
| 121 | agctctccac cgccgcgtgc gcctgcagac gctccgctcg ctgccttctc tcctggcagg |
| 181 | cgctgccttt tctccccgtt aaaagggcac ttgggctgaa ggatcgcttt gagatctgag |
| 241 | gaacccgcag cgctttgagg gacctgaagc tgtttttctt cgttttcctt tgggttcagt |
| 301 | ttgaacggga ggtttttgat cccttttttt cagaatggat tatttgctca tgattttctc |
| 361 | tctgctgttt gtggcttgcc aaggagctcc agaaacagca gtcttaggcg ctgagctcag |
| 421 | cgcggtgggt gagaacggcg gggagaaacc cactcccagt ccaccctggc ggctccgccg |
| 481 | gtccaagcgc tgctcctgct cgtccctgat ggataaagag tgtgtctact tctgccacct |
| 541 | ggacatcatt tgggtcaaca ctcccgagca cgttgttccg tatggacttg gaagccctag |
| 601 | gtccaagaga gccttggaga atttacttcc cacaaaggca acagaccgtg agaatagatg |
| 661 | ccaatgtgct agccaaaaag acaagaagtg ctggaatttt tgccaagcag gaaaagaact |
| 721 | cagggctgaa gacattatgg agaaagactg gaataatcat aagaaaggaa aagactgttc |
| 781 | caagcttggg aaaaagtgta tttatcagca gttagtgaga ggaagaaaaa tcagaagaag |
| 841 | ttcagaggaa cacctaagac aaaccaggtc ggagaccatg agaaacagcg tcaaatcatc |
| 901 | ttttcatgat cccaagctga aaggcaagcc ctccagagag cgttatgtga cccacaaccg |
| 961 | agcacattgg tgacagacct tcggggcctg tctgaagcca tagcctccac ggagagccct |
| 1021 | gtggccgact ctgcactctc caccctggct gggatcagag caggagcatc ctctgctggt |
| 1081 | tcctgactgg caaaggacca gcgtcctcgt tcaaaacatt ccaagaaagg ttaaggagtt |
| 1141 | cccccaacca tcttcactgg cttccatcag tggtaactgc tttggtctct tctttcatct |
| 1201 | ggggatgaca atggacctct cagcagaaac acacagtcac attcgaattc gggtggcatc |
| 1261 | ctccggagag agagagagga aggagattcc acacaggggt ggagtttctg acgaaggtcc |
| 1321 | taagggagtg tttgtgtctg actcaggcgc ctggcacatt tcagggagaa actccaaagt |
| 1381 | ccacacaaag attttctaag gaatgcacaa attgaaaaca cactcaaaag acaaacatgc |
| 1441 | aagtaaagaa aaaaaaaaga aagacttttg tttaaatttg taaaatgcaa aactgaatga |
| 1501 | aactgttact accataaatc aggatatgtt tcatgaatat gagtctacct cacctatatt |
| 1561 | gcactctggc agaagtattt cccacattta attattgcct ccccaaactc ttcccacccc |
| 1621 | tgctgcccct tcctccatcc cccatactaa atcctagcct cgtagaagtc tggtctaatg |
| 1681 | tgtcagcagt agatataata ttttcatggt aatctactag ctctgatcca taagaaaaaa |
| 1741 | aagatcatta aatcaggaga ttccctgtcc ttgatttttg gagacacaat ggtatagggt |
| 1801 | tgtttatgaa atatattgaa aagtaagtgt ttgttacgct ttaaagcagt aaaattattt |
| 1861 | tcctttatat aaccggctaa tgaaagaggt tggattgaat tttgatgtac ttattttttt |
| 1921 | atagatattt atattcaaac aatttattcc ttatatttac catgttaaat atctgtttgg |
| 1981 | gcaggccata ttggtctatg tatttttaaa atatgtattt ctaaatgaaa ttgagaacat |
| 2041 | gctttgtttt gcctgtcaag gtaatgactt tagaaaataa atattttttt ccttactgta |
| 2101 | aaaaaaaaaa aaaaaaa |

The cDNA sequence of human tissue factor pathway inhibitor (TFPI; Accession No. NM_{—}001032281 (1166 by mRNA) is (SEQ ID NO:17):

| | |
|---|---|
| 1 | attcccaact gccagtgatc tctgaagccg actctgaggc tccctctttg ctctaacaga |
| 61 | cagcagcgac tttaggctgg ataatagtca aattcttacc tcgctctttc actgctagta |
| 121 | agatcagatt gcgtttcttt cagttactct tcaatcgcca gtttcttgat ctgcttctaa |
| 181 | aagaagaagt agagaagata aatcctgtct tcaatacctg gaaggaaaaa caaaataacc |
| 241 | tcaactccgt tttgaaaaaa acattccaag aactttcatc agagatttta cttagatgat |
| 301 | ttacacaatg aagaaagtac atgcactttg ggcttctgta tgcctgctgc ttaatcttgc |
| 361 | ccctgcccct cttaatgctg attctgagga agatgaagaa cacacaatta tcacagatac |
| 421 | ggagttgcca ccactgaaac ttatgcattc attttgtgca ttcaaggcgg atgatggccc |
| 481 | atgtaaagca atcatgaaaa gatttttctt caatattttc actcgacagt gcgaagaatt |
| 541 | tatatatggg ggatgtgaag gaaatcagaa tcgatttgaa agtctggaag agtgcaaaaa |
| 601 | aatgtgtaca agagataatg caaacaggat tataaagaca acattgcaac aagaaaagcc |
| 661 | agatttctgc tttttggaag aagatcctgg aatatgtcga ggttatatta ccaggtattt |
| 721 | ttataacaat cagacaaaac agtgtgaacg tttcaagtat ggtggatgcc tgggcaatat |
| 781 | gaacaatttt gagacactgg aagaatgcaa gaacatttgt gaagatggtc cgaatggttt |
| 841 | ccaggtggat aattatggaa cccagctcaa tgctgtgaat aactccctga ctccgcaatc |
| 901 | aaccaaggtt cccagccttt ttgttacaaa agaaggaaca aatgatggtt ggaagaatgc |
| 961 | ggctcatatt taccaagtct ttctgaacgc cttctgcatt catgcatcca tgttctttct |
| 1021 | aggattggat agcatttcat gcctatgtta atatttgtgc ttttggcatt tccttaatat |
| 1081 | ttatatgtat acgtgatgcc tttgatagca tactgctaat aaagttttaa tatttacatg |
| 1141 | catagtaaaa aaaaaaaaaa aaaaaa |

The cDNA sequence of human serpin peptidase inhibitor, member 2 (SERPINE2; Accession No. NM_{—}001136530 (2186 by mRNA) is (SEQ ID NO:18):

| | |
|---|---|
| 1 | gttacctctg cctgggcaga gggaggtgac gcgcggtgtg gggaaagcct ttaacttggc |
| 61 | ctctggcagc cgatttaacc cgagcgagca ggtctttgct attttcatca tctgtagaaa |
| 121 | cgggagttgc gaggcggatg agtgactgca ggtcgtcctt ggtggaagga accatgaact |
| 181 | ggcatctccc cctcttcctc ttggcctctg tgacgctgcc ttccatctgc tcccacttca |
| 241 | atcctctgtc tctcgaggaa ctaggctcca acacggggat ccaggttttc aatcagattg |
| 301 | tgaagtcgag gcctcatgac aacatcgtga tctctcccca tgggattgcg tcggtcctgg |
| 361 | ggatgcttca gctgggggcg gacggcagga ccaagaagca gctcgccatg gtgatgagat |
| 421 | acggcgtaaa tggagttggt aaaatattaa agaagatcaa caaggccatc gtctccaaga |
| 481 | agaataaaga cattgtgaca gtggctaacg ccgtgtttgt taagaatgcc tctgaaattg |
| 541 | aagtgccttt tgttacaagg aacaaagatg tgttccagtg tgaggtccgg aatgtgaact |
| 601 | ttgaggatcc agcctctgcc tgtgattcca tcaatgcatg ggttaaaaat gaaaccaggg |
| 661 | atatgattga caatctgctg tccccagatc ttattgatgg tgtgctcacc agactggtcc |
| 721 | tcgtcaacgc agtgtatttc aagggtctgt ggaaatcacg gttccaaccc gagaacacaa |
| 781 | agaaacgcac tttcgtggca gccgacggga aatcctatca agtgccaatg ctggcccagc |
| 841 | tctccgtgtt ccggtgtggg tcgacaagtg cccccaatga tttatggtac aacttcattg |
| 901 | aactgcccta ccacggggaa agcatcagca tgctgattgc actgccgact gagagctcca |
| 961 | ctccgctgtc tgccatcatc ccacacatca gcaccaagac catagacagc tggatgagca |
| 1021 | tcatggtgcc caagagggtg caggtgatcc tgcccaagtt cacagctgta gcacaaacag |
| 1081 | atttgaagga gccgctgaaa gttcttggca ttactgacat gtttgattca tcaaaggcaa |
| 1141 | attttgcaaa aataacaagg tcagaaaacc tccatgtttc tcatatcttg caaaaagcaa |
| 1201 | aaattgaagt cagtgaagat ggaaccaaag cttcagcagc aacaactgca attctcattg |
| 1261 | caagatcatc gcctccctgg tttatagtag acagaccttt tctgtttttc atccgacata |
| 1321 | atcctacagg tgctgtgtta ttcatggggc agataaacaa accctgaaga gtatacaaaa |
| 1381 | gaaaccatgc aaagcaacga ctactttgct acgaagaaag actcctttcc tgcatctttc |
| 1441 | atagttctgt taaatatttt tgtacatcgc ttctttttca aaactagttc ttaggaacag |
| 1501 | actcgatgca agtgtttctg ttctgggagg tattggaggg aaaaaacaag caggatggct |
| 1561 | ggaacactgt actgaggaat gaatagaaag gcttccagat gtctaaaaga ttctttaaac |
| 1621 | tactgaactg ttacctaggt taacaaccct gttgagtatt tgctgtttgt ccagttcagg |
| 1681 | aatttttgtt ttgttttgtc tatatgtgcg gcttttcaga agaaatttaa tcagtgtgac |
| 1741 | agaaaaaaaa atgttttatg gtagctttta ctttttatga aaaaaaaatt atttgccttt |
| 1801 | taaattcttt tcccccatcc ccctccaaag tcttgatagc aagcgttatt ttgggggtag |
| 1861 | aaacggtgaa atctctagcc tctttgtgtt tttgttgttg ttgttgttgt tgttttatat |
| 1921 | aatgcatgta ttcactaaaa taaaatttaa aaaactcctg tcttgctaga caaggttgct |
| 1981 | gttgtgcagt gtgcctgtca ctactggtct gtactccttg gatttgcatt tttgtatttt |
| 2041 | gtacaaagta aaaataaact gttatgagta gtaaaaataa agctatttct ctgctatttg |
| 2101 | aaaatacaat agaagaaact gagcctttta gacattcgtc agcctcttct aataaacctt |
| 2161 | tgtactatgt aaacatcagg aaattc |

The cDNA sequence of human proteoglycan 4, also known as megakaryocyte stimulating factor (PRG4; Accession No. U70136 or NM_{—}005807 (5041 by mRNA) is (SEQ ID NO:19):

| | |
|---|---|
| 1 | gcggccgcga ctattcggta cctgaaaaca acgatggcat ggaaaacact tcccatttac |
| 61 | ctgttgttgc tgctgtctgt tttcgtgatt cagcaagttt catctcaaga tttatcaagc |
| 121 | tgtgcaggga gatgtgggga agggtattct agagatgcca cctgcaactg tgattataac |
| 181 | tgtcaacact acatggagtg ctgccctgat ttcaagagag tctgcactgc ggagctttcc |
| 241 | tgtaaaggcc gctgctttga gtccttcgag agagggaggg agtgtgactg cgacgcccaa |
| 301 | tgtaagaagt atgacaagtg ctgtcccgat tatgagagtt tctgtgcaga agtgcataat |
| 361 | cccacatcac caccatcttc aaagaaagca cctccacctt caggagcatc tcaaaccatc |
| 421 | aaatcaacaa ccaaacgttc acccaaacca ccaaacaaga agaagactaa gaaagttata |
| 481 | gaatcagagg aaataacaga agaacattct gtttctgaaa atcaagagtc ctcctcctcc |
| 541 | tcctcctctt cctcttcttc ttcaacaatt tggaaaatca agtcttccaa aaattcagct |
| 601 | gctaatagag aattacagaa gaaactcaaa gtaaaagata acaagaagaa cagaactaaa |
| 661 | aagaaaccta cccccaaacc accagttgta gatgaagctg gaagtggatt ggacaatggt |
| 721 | gacttcaagg tcacaactcc tgacacgtct accacccaac acaataaagt cagcacatct |
| 781 | cccaagatca caacagcaaa accaataaat cccagaccca gtcttccacc taattctgat |
| 841 | acatctaaag agacgtcttt gacagtgaat aaagagacaa cagttgaaac taaagaaact |
| 901 | actacaacaa ataaacagac ttcaactgat ggaaaagaga agactacttc cgctaaagag |
| 961 | acacaaagta tagagaaaac atctgctaaa gatttagcac ccacatctaa agtgctggct |
| 1021 | aaacctacac ccaaagctga aactacaacc aaaggccctg ctctcaccac tcccaaggag |
| 1081 | cccacgccca ccactcccaa ggagcctgca tctaccacac ccaaagagcc cacacctacc |
| 1141 | accatcaagt ctgcacccac cacccccaag gagcctgcac ccaccaccac caagtctgca |
| 1201 | cccaccactc ccaaggagcc tgcacccacc accaccaagg agcctgcacc caccactccc |
| 1261 | aaggagcctg cacccaccac caccaaggag cctgcaccca ccaccaccaa gtctgcaccc |
| 1321 | accactccca aggagcctgc acccaccacc cccaagaagc ctgccccaac tacccccaag |
| 1381 | gagcctgcac ccaccactcc caaggagcct acacccacca ctcccaagga gcctgcaccc |
| 1441 | accaccaagg agcctgcacc caccactccc aaagagcctg cacccactgc ccccaagaag |
| 1501 | cctgccccaa ctacccccaa ggagcctgca cccaccactc ccaaggagcc tgcacccacc |
| 1561 | accaccaagg agccttcacc caccactccc aaggagcctg cacccaccac caccaagtct |
| 1621 | gcacccacca ctaccaagga gcctgcaccc accactacca agtctgcacc caccactccc |
| 1681 | aaggagcctt cacccaccac caccaaggag cctgcaccca ccactcccaa ggagcctgca |
| 1741 | cccaccaccc ccaagaagcc tgccccaact acccccaagg agcctgcacc caccactccc |
| 1801 | aaggaacctg cacccaccac caccaagaag cctgcaccca ccgctcccaa agagcctgcc |
| 1861 | ccaactaccc ccaaggagac tgcacccacc acccccaaga agctcacgcc caccaccccc |
| 1921 | gagaagctcg cacccaccac ccctgagaag cccgcaccca ccacccctga ggagctcgca |
| 1981 | cccaccaccc ctgaggagcc cacacccacc acccctgagg agcctgctcc caccactccc |
| 2041 | aaggcagcgg ctcccaacac ccctaaggag cctgctccaa ctacccctaa ggagcctgct |
| 2101 | ccaactaccc ctaaggagcc tgctccaact acccctaagg agactgctcc aactacccct |
| 2161 | aaagggactg ctccaactac cctcaaggaa cctgcaccca ctactcccaa gaagcctgcc |
| 2221 | cccaaggagc ttgcacccac caccaccaag gagcccacat ccaccacctc tgacaagccc |
| 2281 | gctccaacta cccctaaggg gactgctcca actaccccta aggagcctgc tccaactacc |
| 2341 | cctaaggagc ctgctccaac tacccctaag gggactgctc caactaccct caaggaacct |
| 2401 | gcacccacta ctcccaagaa gcctgccccc aaggagcttg cacccaccac caccaagggg |
| 2461 | cccacatcca ccacctctga caagcctgct ccaactacac ctaaggagac tgctccaact |
| 2521 | acccccaagg agcctgcacc cactaccccc aagaagcctg ctccaactac tcctgagaca |
| 2581 | cctcctccaa ccacttcaga ggtctctact ccaactacca ccaaggagcc taccactatc |
| 2641 | cacaaaagcc ctgatgaatc aactcctgag ctttctgcag aacccacacc aaaagctctt |
| 2701 | gaaaacagtc ccaaggaacc tggtgtacct acaactaaga ctcctgcagc gactaaacct |
| 2761 | gaaatgacta caacagctaa agacaagaca acagaaagag acttacgtac tacacctgaa |
| 2821 | actacaactg ctgcacctaa gatgacaaaa gagacagcaa ctacaacaga aaaaactacc |
| 2881 | gaatccaaaa taacagctac aaccacacaa gtaacatcta ccacaactca agataccaca |
| 2941 | ccattcaaaa ttactactct taaaacaact actcttgcac ccaaagtaac tacaacaaaa |
| 3001 | aagacaatta ctaccactga gattatgaac aaacctgaag aaacagctaa accaaaagac |
| 3061 | agagctacta attctaaagc gacaactcct aaacctcaaa agccaaccaa agcacccaaa |
| 3121 | aaacccactt ctaccaaaaa gccaaaaaca atgcctagag tgagaaaacc aaagacgaca |
| 3181 | ccaactcccc gcaagatgac atcaacaatg ccagaattga accctacctc aagaatagca |
| 3241 | gaagccatgc tccaaaccac caccagacct aaccaaactc caaactccaa actagttgaa |
| 3301 | gtaaatccaa agagtgaaga tgcaggtggt gctgaaggag aaacacctca tatgcttctc |
| 3361 | aggccccatg tgttcatgcc tgaagttact cccgacatgg attacttacc gagagtaccc |
| 3421 | aatcaaggca ttatcatcaa tcccatgctt tccgatgaga ccaatatatg caatggtaag |
| 3481 | ccagtagatg gactgactac tttgcgcaat gggacattag ttgcattccg aggtcattat |
| 3541 | ttctggatgc taagtccatt cagtccacca tctccagctc gcagaattac tgaagtttgg |
| 3601 | ggtattcctt cccccattga tactgttttt actaggtgca actgtgaagg aaaaactttc |
| 3661 | ttctttaagg attctcagta ctggcgtttt accaatgata taaaagatgc agggtacccc |
| 3721 | aaaccaattt tcaaaggatt tggaggacta actggacaaa tagtggcagc gctttcaaca |
| 3781 | gctaaatata agaactggcc tgaatctgtg tattttttca agagaggtgg cagcattcag |
| 3841 | cagtatattt ataaacagga acctgtacag aagtgccctg gaagaaggcc tgctctaaat |
| 3901 | tatccagtgt atggagaaat gacacaggtt aggagacgtc gctttgaacg tgctatagga |
| 3961 | ccttctcaaa cacacaccat cagaattcaa tattcacctg ccagactggc ttatcaagac |
| 4021 | aaaggtgtcc ttcataatga agttaaagtg agtatactgt ggagaggact tccaaatgtg |
| 4081 | gttacctcag ctatatcact gcccaacatc agaaaacctg acggctatga ttactatgcc |
| 4141 | ttttctaaag atcaatacta taacattgat gtgcctagta gaacagcaag agcaattact |
| 4201 | actcgttctg ggcagacctt atccaaagtc tggtacaact gtccttagac tgatgagcaa |
| 4261 | aggaggagtc aactaatgaa gaaatgaata ataaattttg acactgaaaa acattttatt |
| 4321 | aataaagaat attgacatga gtataccagt ttatatataa aaatgttttt aaacttgaca |
| 4381 | atcattacac taaaacagat ttgataatct tattcacagt tgttattgtt tacagaccat |
| 4441 | ttaattaata tttcctctgt ttattcctcc tctccctccc attgcatggc tcacacctgt |
| 4501 | aaaagaaaaa agaatcaaat tgaatatatc ttttaagaat tcaaaactag tgtattcact |
| 4561 | taccctagtt cattataaaa aatatctagg cattgtggat ataaaactgt tgggtattct |
| 4621 | acaacttcaa tggaaattat tacaagcaga ttaatccctc tttttgtgac acaagtacaa |
| 4681 | tctaaaagtt atattggaaa acatggaaat attaaaattt tacactttta ctagctaaaa |
| 4741 | cataatcaca aagctttatc gtgttgtata aaaaaattaa caatataatg gcaataggta |
| 4801 | gagatacaac aaatgaatat aacactataa cacttcatat tttccaaatc ttaatttgga |
| 4861 | tttaaggaag aaatcaataa atataaaata taagcacata tttattatat atctaaggta |
| 4921 | tacaaatctg tctacatgaa gtttacagat tggtaaatat cacctgctca acatgtaatt |
| 4981 | atttaataaa actttggaac attaaaaaaa taaattggag gcttaaaaaa aaaaaaaaaa |
| 5041 | a |

Sequences are available to an art worker, for example a cDNA sequence of defensin related cryptdin 3 (DEFCR3) is provided at Accession No. NM_{—}007850); a cDNA sequence for collagen, type XVIII, alpha 1 (COL18A1) is provided at Accession No. NM_{—}130444; a cDNA sequence for bone gamma-carboxyglutamate protein (BGLAP) is provided at Accession No. NM_{—}199173 and XO4143; a cDNA sequence for collage, type VIII, alpha 1 (COL8A1) is provided at NM_{—}001850 and AF170702, a cDNA sequence for proteoglycan 1, secretory granule (PRG1; also known as serglycin) is provided at NM_{—}002727 or BC015516, a cDNA sequence for inhibin, beta A (INHBA) is provided at NM_{—}002192, a cDNA sequence for latent transforming growth factor beta binding protein 2 (LTBP2) is provided at NM_{—}000428.

Of the differentially expressed genes, two have been identified as factors that support HSC maintenance in vitro. Delta-like 1 homolog (Dlk-1), expressed 100-fold more highly in UG26-1B6 than EL08-1D2 cells is in part responsible for the maintenance of HSC in contact with the AFT024 feeder [48]. Insulin-like growth factor binging protein 3 (Igfb3) modulates the effect of insulin growth factor (Igf) proteins. Insulin growth factor 1 (Igfl) and Insulin-like growth factor binding protein 2 (Igbp2) have been identified to be produced by stromal cells and be in part responsible for the maintenance of HSC in these systems [5, 49, 50].

Effect of Secreted Factors More Highly Expressed in UG26-1B6 than EL08-1D2 and AFT024 Feeders, on HSC Maintenance/Expansion.

The role of proteins encoded by the other identified genes on HSC regulation is unknown. Initial experiments were performed on several of these genes to evaluate their effect on HPC and HSC in vitro.

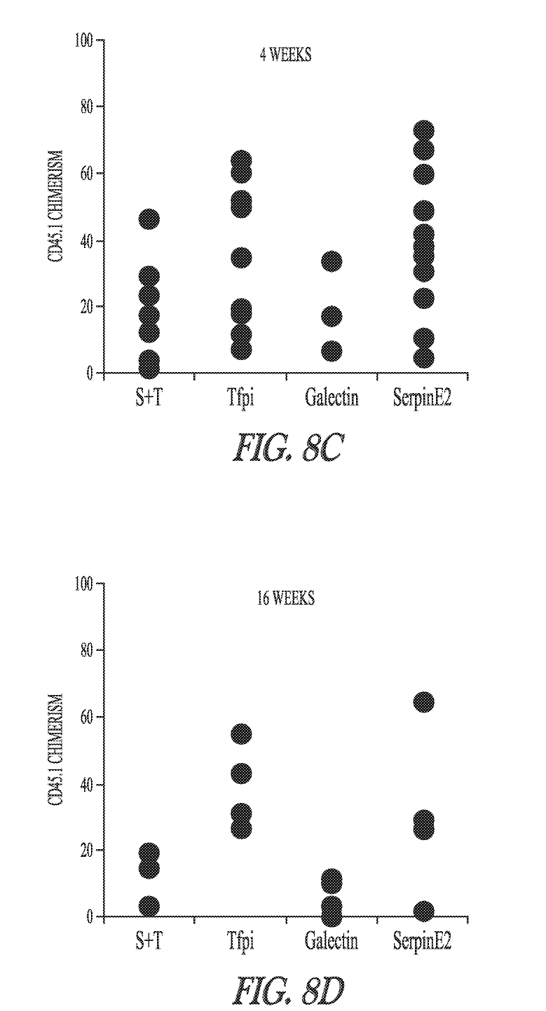

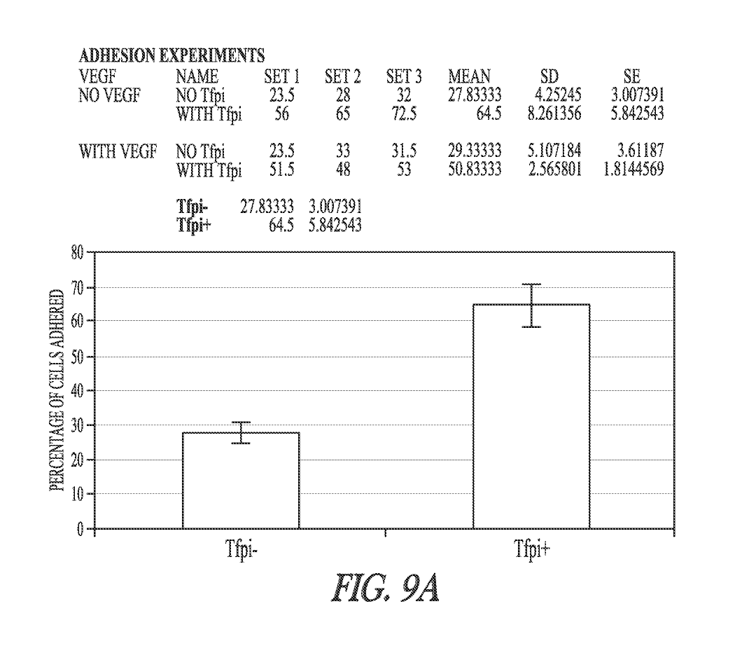

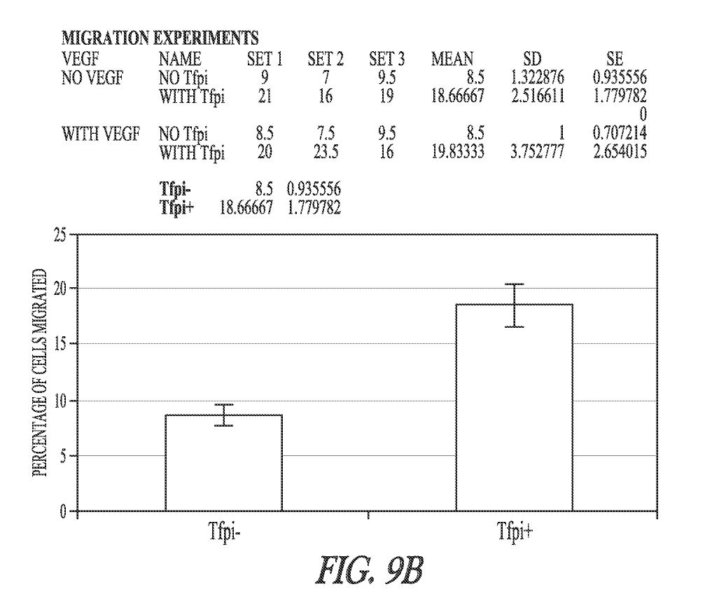

50 c-kit+/Sca-1+/Lin− (KLS) murine BM cells were cultured in U-bottom 96 well plates for 5 days in serum free medium supplemented with 100 ng/mL SCF, 50 ng/mL Tpo, and 100 ng/mL Tfpi, 5 mg/ml galectin or 5 mg/ml SerpinE2. No significant differences were found in total cell expansion of KLS cells when Tfpi, SerpinE2, or Galectin were added (FIG. 8A). No significant differences were found in the number of CFC recovered after 5 days from cultures supplemented with SCF and Tpo with or without Tfpi, galectin, or SerpinE2 (FIG. 8B).

Competitive repopulation studies were performed with 200 freshly isolated KLS cells or culture progeny of 200 KLS cells from CD45.1+ mice, cultured with SCF and Tpo with or without Tfpi, galectin, or SerpinE2, with 10⁵ BM cells from CD45.2+ mice in lethally irradiated CD45.2+ recipient. At 4 and 16 weeks PB was analyzed for multi-lineage engraftment from the CD45.1+ KLS cells. Recipients were considered engrafted if >1% CD45.1+ cells were detected with contribution to the myeloid, B-lymphoid and T-lymphoid lineages. In our studies in 5 mice, we show that addition of galectin to the culture does not affect hematopoietic reconstitution from KLS cells.

Transplantation of progeny from cultures to which Tfpi was added resulted in similar levels of engraftment in 6 mice at 4 weeks compared to when no Tfpi was added (13.03%±5.29% and 17.34%±9.84%, respectively). Contribution to both the myeloid and lymphoid lineage was similar at 4 weeks. By contrast, significantly greater levels of engraftment from KLS cells cultured with Tfpi compared to without Tfpi (38.65%±12.78% and 3.4%±0.85%, p-value=0.01) (FIGS. 8C&D) were seen at 16 weeks following transplantation (n=4). The donor-derived cells contributed equally to myeloid and lymphoid lineages. With these results we show that addition of Tfpi enhances the maintenance of long-term repopulating HSC (LTR-HSC) and this is useful for HSC expansion.

Transplantation of KLS cells cultured with Tpo and SCF with in addition SerpinE2 yielded higher levels of CD45.1 derived reconstitution at 4 weeks compared to cells treated with SCF and Tpo alone (32.04%±15.34% and 17.34%±9.84%, respectively (n=5)). Engraftment of myeloid and lymphoid compartment showed normal lineage distribution from the donor derived cells. Similarly at 12-16 weeks post-transplantation KLS cells cultured in the presence of SerpinE2 yielded higher CD45.1 derived engraftment compared to cells in the presence of SCF and Tpo alone (24.7±25.6% and 8.7±7.3%, respectively).

### Discussion

To identify possible novel secreted factors that support HSC maintenance and/or expansion, stromal feeders that support (UG26-1B6 cells) or do not support (EL08-1D2 cells) maintenance of murine LTR-HSC when cultured in transwells above the irradiated feeders were used. A transcriptome analysis of these feeders demonstrated several of these genes encoding proteins classified by the Gene Ontology classification to be present in the extracellular space, 15 of which have previously not been shown to affect HSC.

Studies done with Wnt5a, previously shown to have an effect on HSC, are described herein below. Among the other differentially expressed secreted factors, Dlk1 and Igfbp3 have been shown to influence HSC maintenance/expansion.

The effect of 4/18 of the identified proteins, specifically Galectin, Tfpi and SerpinE2, was tested on HSC maintenance/expansion ex vivo, using an established serum-free stroma-free culture system of KLS cells supplemented with SCF and Tpo [5]. Tfpi is an anti-coagulant in vivo, but a role for Tfpi in hematopoiesis has not been described [51, 52]. There is also no published data demonstrating a role for Galectin or SerpinE2 in hematopoiesis. Initial results suggest that Tfpi may increase the number of HSC that engraft mice at 4 months, whereas reconstitution by KLS cells cultured with SerpinE2 was increased at 3 weeks and 3-4 months. These findings suggest that more than one of the identified factors may be responsible for maintaining HSC in stromal non-contact cultures and the HSC microenvironment is a complex network of signals responsible for cell fate decisions.

A number of genes that are differentially expressed between the two feeders categorized as unknown, can also encode for proteins in the extracellular space (Table 4). These gene products can also be factors involved in hematopoiesis.

TABLE 4
Genes expressed by UG26-1B6 with unknown or uncategorized cellular location.

| | | Fold |
|---|---|---|
| Gene Symbol | Gene | Change |
| PEG12 | paternally expressed 12 | 28 |
| FLJ20701 | hypothetical protein FLJ20701 | 17 |
| SPBC24 | spindle pole body component 24 homolog | 14 |
| PPIL5 | peptidylprolyl isomerase (cyclophilin)-like 5 | 11 |
| FKSG14 | leucine zipper protein FKSG14 | 11 |
| C15ORF23 | chromosome 15 open reading frame 23 | 11 |
| C10ORF3 | centrosomal protein 55kDa | 10 |
| ARHGAP11A | Rho GTPase activating protein 11A | 10 |
| DHFR | dihydrofolate reductase | 9 |
| CDCA5 | cell division cycle associated 5 | 9 |
| SHCBP1 | SHC SH2-domain binding protein 1 | 9 |
| CDCA3 | cell division cycle associated 3 | 9 |
| SPBC25 | spindle pole body component 25 homolog | 9 |
| IRAK3 | interleukin-1 receptor-associated kinase 3 | 7 |
| ASPM | asp (abnormal spindle)-like, microcephaly associated | 7 |
| FLJ20364 | hypothetical protein FLJ20364 | 6 |
| UBE2T | ubiquitin-conjugating enzyme E2T (putative) | 6 |
| ASF1B | ASF1 anti-silencing function 1 homolog B | 6 |
| PSF1 | DNA replication complex GINS protein PSF1 | 6 |
| RAPGEF5 | Rap guanine nucleotide exchange factor (GEF) 5 | 5 |
| CGREF1 | cell growth regulator with EF-hand domain 1 | 5 |
| CHTF18 | CTF18, chromosome transmission fidelity factor 18 homolog | 5 |
| DTYMK | deoxythymidylate kinase | 5 |
| A130040M12RIK | RIKEN cDNA A130040M12 gene | 5 |
| ELMO1 | engulfment and cell motility 1 | 4 |
| GALNT13 | UDP-N-acetyl-alpha-D-galactosamine:polypeptide N- | 4 |
| | acetylgalactosaminyltransferase 13 | |
| HS3ST6 | heparan sulfate (glucosamine) 3-O-sulfotransferase 6 | 4 |
| FBXO42 | F-box protein 42 | 4 |
| WRNIP1 | Werner helicase interacting protein 1 | 4 |
| NUDT7 | nudix (nucleoside diphosphate linked moiety X)-type motif 7 | 4 |
| STAF42 | transcriptional adaptor 1 (HFI1 homolog, yeast)-like | 4 |
| ATAD2 | ATPase family, AAA domain containing 2 | 4 |
| TLL1 | tolloid-like 1 | 4 |
| LOC93081 | hypothetical protein BC015148 | 4 |
| CDCA4 | cell division cycle associated 4 | 3 |
| BOK | BCL2-related ovarian killer | 3 |
| BRMS1 | breast cancer metastasis suppressor 1 | 3 |
| NKD2 | naked cuticle homolog 2 | 3 |
| LYPLA1 | lysophospholipase I | 3 |
| DDIT4L | DNA-damage-inducible transcript 4-like | 3 |
| SMS | spermine synthase | 3 |
| FLJ22794 | FLJ22794 protein | 3 |
| PKMYT1 | protein kinase, membrane associated tyrosine/threonine 1 | 3 |
| ACYP1 | acylphosphatase 1, erythrocyte (common) type | 3 |
| EYA4 | eyes absent homolog 4 | 3 |
| NUBP1 | nucleotide binding protein 1 | 3 |
| KIAA1794 | KIAA1794 | 3 |
| HN1 | hematological and neurological expressed 1 | 3 |
| CHC1L | regulator of chromosome condensation (RCC1) and BTB | 3 |
| | (POZ) domain containing protein 2 | |
| FLJ12443 | acyltransferase like 2 | 3 |
| CTNNBIP1 | catenin, beta interacting protein 1 | 3 |
| FLJ38973 | hypothetical protein FLJ38973 | 3 |
| NUDT1 | nudix (nucleoside diphosphate linked moiety X)-type motif 1 | 3 |
| B3GALT6 | UDP-Gal:betaGal beta 1,3-galactosyltransferase polypeptide | 2 |
| | 6 | |
| LOC221955 | KCCR13L | 2 |
| SLC43A3 | solute carrier family 43, member 3 | 2 |
| FAM57A | family with sequence similarity 57, member A | 2 |
| PIP5KL1 | phosphatidylinositol-4-phosphate 5-kinase-like 1 | 2 |
| MAGEH1 | melanoma antigen family H, 1 | 2 |
| CHPT1 | choline phosphotransferase 1 | 2 |
| COMMD10 | COMM domain containing 10 | 2 |
| FLJ3798 | hypothetical protein FLJ13798 | 2 |
| DKFZP762E1312 | hypothetical protein DKFZp762E1312 | 2 |
| C9ORF76 | chromosome 9 open reading frame 76 | 2 |
| RQCD1 | RCD1 required for cell differentiation 1 homolog | 2 |
| L2HGDH | L-2-hydroxyglutarate dehydrogenase | 2 |
| SOCS5 | suppressor of cytokine signaling 5 | 2 |
| MGC5508 | transmembrane protein 109 | 2 |
| ADK | adenosine kinase | 2 |
| C14ORF130 | chromosome 14 open reading frame 130 | 2 |
| C14ORF106 | chromosome 14 open reading frame 106 | 2 |
| MUTED | muted homolog (mouse) | 2 |
| IRAK4 | interleukin-1 receptor-associated kinase 4 | 2 |
| UBE2S | ubiquitin-conjugating enzyme E2S | 2 |
| ING5 | inhibitor of growth family, member 5 | 2 |
| CXORF53 | BRCA1/BRCA2-containing complex, subunit 3 | 2 |
| MTBP | Mdm2, transformed 3T3 cell double minute 2, p53 binding | 2 |
| | protein (mouse) binding protein | |
| PRPS1 | phosphoribosyl pyrophosphate synthetase 1 | 2 |
| C10ORF119 | chromosome 10 open reading frame 119 | 2 |
| CASP8AP2 | CASP8 associated protein 2 | 2 |

### Example 3

### Wnt5a is Capable of Maintaining Hematopoietic Stem Cells

### Introduction

Many studies have found that cytokines and/or growth factors alone fail to support the maintenance of CR-LTR-HSC in vitro. More recently, studies have evaluated the role of morphogens known to play a role in early embryo development in HSC self-renewal, maintenance and differentiation. These include members of the hedgehog (HH) family, the transforming growth factor (TGF) family, and Wnts.

In the previous two examples it was demonstrated that the UG26-1B6 cell line derived from the urogenital ridge of E10.5 mouse embryos possesses the unique ability to maintain CR-LTR-HSC for 3 weeks cultured in transwells above the feeder without addition of exogenous cytokines. Subsequent studies wherein we compared the transcriptome of UG26-1B6, and EL08-1D2 cells using Affymetrix gene arrays identified Wnt5a as one of the genes coding for a secreted factor significantly higher expressed in UG26-1B6 cells.

Wnt proteins are grouped into different categories based on the downstream signal pathways that are activated. The best understood pathway is the canonical pathway, wherein ligands bind to their respective receptors, and stabilize β-catenin β-catenin then translocates to the nucleus leading to activation of the Tcf/Lef transcription factors. In the absence of Wnt, β-catenin is found in a complex with among others, adenomatous polyposis coli (APC) and glycogen synthase kinase-3 β (GSK3 β). GSK3 β phosphorylates β-catenin, targeting β-catenin for ubiquitination and degradation by the proteasome. Wnt binding to frizzled along with its co-receptor low-density lipoprotein receptor related protein (LRP) leads to phosphorylation of Dishevelled 2 (Dv12), enabling Dv12 to inhibit the ability of GSK3 β ability to phosphorylate β-catenin A typical example of Wnts that affect the canonical pathway is Wnt3a, which has been shown to enhance survival of LTR-HSC ex vivo. By contrast, Wnt5a has been categorized as a non-canonical Wnt. Wnt5a mediates its activity by binding to the orphan tyrosine kinase, Ror2 and thus inhibits the β-catenin dependent canonical pathways. However, a recent study has demonstrated that Wnt5a may also activate the canonical pathway by binding to the Frizzled 4 receptor.

Wnt proteins are expressed in the developing embryo in sites of hematopoiesis like the fetal liver168 as well as the osteoblastic niche in postnatal BM160, and there is mounting evidence that Wnts affect the self renewal of HSC in postnatal life. For instance, Reya, et. al. demonstrated that Wnt3a supports HSC self-renewal in vitro. Stromal cells transfected with Wnt1, Wnt2b, Wnt5a, or Wnt10b support proliferation of fetal liver cells, or CD34+ human BM cells. Intraperitoneal injection of Wnt5a conditioned media increased engraftment potential of CD34+ human cells. In mice where Dkk1, an inhibitor of the Wnt pathway, is over-expressed in the osteoblastic niche, a reduced activation of the Tcf/Lef transcription factors in HSC was seen. Although HSC from Dkk1 transgenic mice could reconstitute hematopoiesis in primary mice, a significant decrease in reconstituting capacity after serial BM transplantation was noted. These studies suggest that loss of activation of the canonical pathway by Wnts in the niche results in the premature loss of self-renewal activity. Nevertheless, it remains unclear what the precise role is of β-catenin in hematopoiesis. Mice lacking β-catenin have no defects in HSC maintenance and survival, whereas constitutive active β-catenin leads to defects in multilineage potential of HSC in vivo.

Herein it was tested whether Wnt5a, which is significantly higher expressed in UG26-1B6 cells than EL08-1D2 cells, is responsible for the ability of UG26-1B6, but not EL08-1D2, cells to support CR-LTR-HSC in non-contact culture. When Wnt5a was added to EL08-1D2 cultures improved maintenance of STR-HSC and to a lesser extent CR-LTR-HSC was observed, whereas addition of an anti-Wnt5a antibody to UG26-1B6 non-contact cultures inhibited maintenance of STR-HSC and CR-LTR-HSC. Wnt5a inhibited the canonical pathway in EL08-1D2 cells, but appeared to activate the canonical pathway in KLS-CD34− cells that express Frizzled 4, but not Ror2 receptors. When Wnt5a was added to a non-stroma dependent 5-day culture system also containing SCF and Tpo, a significant increase in maintenance and perhaps even expansion of CR-LTR-HSC was noted.

### Results

Wnt5a is More Highly Expressed in UG26-1B6 than EL08-1D2 Stromal Cells. Herein the expression of Wnt proteins in UG26-1B6 and EL08-1D2 cells was evaluated by RT-qPCR. Although many Wnt genes are expressed, the level of expression in general is low, except for Wnt5a that was significantly higher expressed in UG26-1B6 than EL08-1D2 cells. To confirm the differential expression further, western blot analysis was preformed. As Wnt5a is a secreted protein it was initially tested whether the protein could be quantified in stromal cell line conditioned medium. However, this was unsuccessful. Nevertheless, when the protein level was measured in total cell lysates, significantly higher levels of Wnt5a protein were detected in UG26-1B6 than EL08-1D2 cells.

Addition of Wnt5a to EL08-1D2-Non-Contact Cultures Increases Maintenance of CR-LTR-HSC

To determine if Wnt5a produced by UG26-1B6 cells is responsible for the maintenance of LTR-HSC cultured in transwells above the feeder, CD45.1+ Lin− BM was cultured in transwells above the EL08-1D2 or UG26-1B6 cells with or without 10 ng/ml Wnt5a, added weekly for the 3-week culture period. As control, Lin− BM cells were also cultured in direct contact with the feeders with or without Wnt5a. Week 3 progeny was enumerated, and progeny of 10⁴ Lin− BM cells harvested from the different cultures were co-transplanted with 10⁵ BM cells from CD45.2+ mice in lethally irradiated CD45.2+ mice. Addition of Wnt5a to EL08-1D2 or UG26-1B6-contact cultures did not affect the maintenance of CR-LTR-HSC after 3 weeks.

Addition of Wnt5a to EL08-1D2 or UG26-1B6 non-contact cultures did not affect cell expansion at 3 weeks. Also, addition of Wnt5a to UG26-1B6-non-contact cultures did not affect the percentage of mice engrafted with progeny of the CD45.1+ cells, nor the levels of engraftment seen from the CD45.1+ donor cells. By contrast, when Lin− CD45.1+ cells were cultured in EL08-1D2-non-contact cultures supplemented with 10 ng/ml Wnt5a, 9/13 mice showed multilineage CD45.1+ cell derived engraftment at 4 months, while no CD45.1+ cells were detected in animals grafted with cells cultured in EL08-1D2-non-contact cultures not supplemented with Wnt5a. As noticed for cells cultured in EL08-1D2-contact cultures (with or without Wnt5a), engraftment seen with cells from EL08-1D2-non-contact cultures supplemented with Wnt5a was skewed towards the B-lymphoid lineage (p-value=0.002 & 0.02, B-lymphoid lineage compared to fresh uncultured cells and UG26-1B6 non-contact cultures supplemented with Wnt5a, respectively). When higher concentrations of Wnt5a were added (50 and 100 ng/ml Wnt5a weekly for 3 weeks), no further increase was observed in the frequency of mice engrafted with CD45.1+ progeny, or the relative contribution of CD45.1+ cells to the lymphoid and myeloid lineages.

To further substantiate a role for Wnt5a in maintenance of LTR-HSC in UG26-1B6 non-contact cultures, we added 1 μg/ml-neutralizing antibody to Wnt5a to UG26-1B6-non-contact cultures, weekly for 3 weeks. Anti-Wnt5a inhibited the ability of UG26-1B6 feeders to maintain CR-LTR-HSC, as 0 out of 7 mice were engrafted with CD45.1+ cells at 4 months, whereas 2/5 animals grafted with progeny of UG26-1B6-noncontact cultures treated with 1 μg/ml anti-IgG control antibodies and 3/5 animals grafted with cells maintained in UG26-1B6-non-contact cultures without antibody addition were engrafted with CD45.1+ progeny. These studies demonstrate that Wnt5a is one of the secreted factors in UG26-1B6 responsible for maintenance of CR-LTR-HSC. Wnt5a can act either directly onto the HSC or indirectly by altering production of other factors by the feeder.

Wnt5a Inhibits the Canonical Pathway in EL08-1D2 Cells, but Activates the β-Catenin Pathway in KLS and CD34-KLS Cells

To further determine the mechanism of action of Wnt5a in the culture system described herein, the activation of signaling pathways downstream of Wnts was examined. Wnt5a is considered a member of the non-canonical Wnt signaling proteins, which following binding to the orphan receptor Ror2 inhibits the β-catenin activation. However, a recent study has suggested that Wnt5a may also bind to Fzd4 and its co-receptor Lrp5 leading to activation of the β-catenin pathway.

RT-qPCR revealed that EL08-1D2 and UG26-1B6 cells expressed receptors thought to be targets of Wnt5a including Fzd-4 (as well as Fzd-4, -5, and -7) and Ror-2. To evaluate if Wnt5a activates the canonical or non-canonical signal pathway in EL08-1D2 or UG26-1B6 cells, the feeders were grown to confluence, irradiated at 25Gy and then exposed to 100 ng/ml Wnt5a. As a positive control for β-catenin activation NIH3T3 cells were incubated with 100 ng/ml Wnt3a. As expected, treatment of NIH3T3 cells with Wnt3a induced an increase in activated β-catenin and an increase in disheveled-2 (Dvl-2). Treatment of UG26-1B6 with 100 ng/mL Wnt5a did not affect the total levels of β-catenin protein, levels of activated β-catenin or the levels of Dvl-2. By contrast, incubation of EL08-1D2 with Wnt5a resulted in a decrease of both total β-catenin and activated β-catenin, but no difference in the level of Dvl-2. Transcripts of β-catenin target genes, such as CyclinD1 and c-Myc were not altered in EL08-1D2 treated with Wnt5a compared with cells not treated with Wnt5a. Thus, Addition of Wnt5a to UG26-1B6 cells did not lead to inactivation or activation of the canonical signaling pathway, likely reflecting the fact that significant amounts of Wnt5a are already present in this culture system. By contrast, Wnt5a inhibits the β-catenin pathway in EL08-1D2 cells consistent with non-canonical signaling in this cell line.

Similar studies in KLS and CD34-KLS cells were then performed. Microarray studies by Zhong, et. al. showed that Fzd4 distinguishes LTR-HSC from hematopoietic progenitor cells (HPC) and STR-HSC. RT-qPCR was used to assess the level of expression of Fzd2, Fzd4, Fzd5, Fzd7, and Ror2 in total BM cells, Lin− BM cells, and HSC-enriched BM-derived KLS cells. Fzd2 was not expressed in either BM, Lin− BM, or KLS cells. By contrast, Fzd5 and Fzd7 were expressed in all three cell populations with higher expression of Fzd7 in Lin− BM cells and KLS cells than total BM cells. Ror2 could not be detected in any of the three cell populations. Fzd4 was solely expressed in the KLS population.

KLS cells were stained with an anti-Fzd4 antibody and it was found that Fzd4 was expressed by a small subpopulation of KLS cells (12.9±12.0%). Although the KLS population is enriched for HSC it still contains a mixture of HPC, STR-HSC and LTR-HSC 58. To determine if Fzd4 is expressed on the LTR-HSC subpopulation of KLS cells, CD34-KLS cells were sorted, which are more highly enriched in LTR-HSC, into a drop of serum-free media and performed single-cell immunostaining for Fzd4. 90±5% of CD34-KLS cells stained positive for Fzd4. KLS cells were also subfractionated using the anti-Fzd4 antibody and performed competitive repopulation assays. 100 Fzd4+ KLS or 100 Fzd4—KLS cells from CD45.1+ mice were co-transplanted with 10⁵ BM cells from CD45.2+ mice in lethally irradiated CD45.2+ mice. Only 1/7 mice transplanted with Fzd4—KLS showed CD45.1+ cell engraftment whereas 8/8 mice transplanted with Fzd4+ KLS cells were repopulated with CD45.1+ cells (p=0.05). This data demonstrates that CR-LTR-HSC express Fzd4.

Next it was determined whether Wnt5a signals via the canonical or non-canonical pathway in KLS and CD34-KLS cells. Treatment of KLS cells for 3 hours with 100 ng/ml Wnt5a revealed an increase in the level of the activated form of β-catenin, suggesting that Wnt5a can activate the canonical signal pathway in KLS cells. To further substantiate this, the effect of Wnt5a on CD34-KLS cells was also evaluated.

50 to 100 CD34-KLS were sorted into a drop of serum-free media and the cells were stimulated for 3 hours in the presence of SCF and Tpo with or without Wnt5a. We then examined total β-catenin accumulation and activated β-catenin in CD34-KLS. Addition of SCF and Tpo lead to a slight accumulation of total β-catenin and non-phosphorylated activated β-catenin (ABC). However, the addition of SCF, Tpo, and Wnt5a lead to a significant increase in total 13-catenin as well as non-phosphorylated ABC, suggesting that addition of Wnt5a to CD34-KLS leads to stabilization and accumulation of β-catenin in the cells.

Addition of Wnt5a to Stroma-Free Serum-Free Cultures of KLS Cells Supplemented with SCF and Tpo Enhances LT-HSC Potential.

Next it was tested whether Wnt5a has a direct effect on CR-LTR-HSC maintenance/expansion in vitro. 50 KLS cells per well were cultured in serum-free medium without feeder cells but with 100 ng/ml thrombopoietin (Tpo), 50 ng/ml stem cell factor (SCF), and with or without 100 ng/ml Wnt5a for 5 days. Total cell number on day 5 was increased approximately 40-fold in cultures with or without Wnt5a. The total number of CFC was similar on day 5 in cultures with or without Wnt5a.

Next 200 freshly isolated KLS cells or progeny of 200 KLS cells cultured in serum-free, stroma-free cultures for 5 days with SCF and Tpo with or without Wnt5a were transplanted together with 10⁵ BM cells from CD45.2+ cells in lethally irradiated CD45.2+ mice. Four weeks after transplantation, the percent CD45.1+ cells present in blood of mice that received KLS cells cultured with or without Wnt5a was similar (16.13±10.09% and 12.78±9.03%, p-value=0.3). However, KLS progeny cultured with Wnt5a generated significantly higher levels of myeloid cells than KLS cells cultured without Wnt5a (8.28±9.56% and 22.56±21.89%) (p<0.01). A similar pattern was seen at 8 and 16 weeks after transplantation.

10⁶ mononuclear BM cells from two of the primary recipients grafted 4 months previously with 200 CD45.1+ KLS cells cultured with Tpo and SCF with or without Wnt5a, were transplanted into 4 each secondary CD45.2+ recipients. Chimerism of CD45.1+ cells was evaluated in the PB 14 weeks after transplantation. Limited to no engraftment was seen in secondary recipients grafted with cells harvested from primary recipients transplanted with KLS progeny cultured with SCF and Tpo alone (2/3 secondary recipients grafted with cells from a primary recipient with 22.8% CD45.1 chimerism only had an average of 1.8±1.2% engraftment, and 0/4 secondary recipients grafted with BM derived from a primary recipient with 8.2% CD45.1 chimerism). By contrast the mean CD45.1+ cell percentage in the peripheral blood of the 4 recipients grafted with BM from a primary recipient reconstituted with 27.1% CD45.1 cells derived from SCF, Tpo and Wnt5a treated KLS cells was 62.1±22.0%. 3/4 animals recipients of BM from the primary recipient reconstituted with 6.5% CD45.1 cells derived from SCF, Tpo and Wnt5a treated KLS cells had multilineage reconstitution with CD45.1+ cells.

### Discussion

The nature of signals that govern HSC self-renewal and differentiation is still largely unknown. Throughout development, HSC reside in multiple different sites. It is therefore thought that characterization of the microenvironment of these developmentally diverse sites may yield important insights in factors that regulate HSC behavior. Stromal cell lines have been generated from the different hematopoietic supportive organs, including yolk sac, AGM, embryonic liver, fetal liver, fetal BM and adult BM with differing abilities to support murine or human HSC. It was shown herein that the AGM-derived UG26-1B6 feeder supports HSC when cultured separated from the feeder by a transwell, whereas the embryonic liver derived EL08-1D2 feeder does not. Based on transcriptome analysis of both feeders, Wnt5a was identified as one of the secreted factors highly differentially expressed between these feeders. Herein it was demonstrated that Wnt5a is at least in part responsible for the ability of UG26-1B6 to support CR-LTR-HSC in non-contact cultures.

Addition of Wnt5a to EL08-1D2-non-contact cultures resulted in maintenance of CR-LTR-HSC whereas addition of an anti-Wnt5a antibody to UG26-1B6 non-contact cultures inhibited the ability of UG26-1B6 cells to maintain CR-LTR-HSC. Of note hematopoietic repopulation in animals receiving cells cultured in contact with EL08-1D2-feeder cells or in transwells above EL08-1D2 cells in the presence of Wnt5a showed skewing to the lymphoid lineage. Another Wnt, Wnt4, is highly expressed in EL08-1D2, but not UG26-1B6 cells. Wnt4 is expressed in the thymus and plays a role in thymocyte development. Mice lacking Wnt4 display a decrease in total number of thymocytes, while maturation is normal. It is thus possible that presence of Wnt4 in EL08-1D2-based cultures induces skewing towards lymphoid committed cells. However, as described in Example 1, mixing studies wherein a combination of 25% UG26-1B6 and 75% EL08-1D2 cells served to support Lin− BM cells in transwells above the feeder, demonstrated robust multilineage engraftment without skewing to the lymphoid lineage from cells cultured above the mixed feeders. This argues against an inhibitor of HSC maintenance, or perhaps an inducer of HSC differentiation to be responsible for the poor maintenance of HSC in EL08-1D2-non-contact cultures, but for the absence of one or more factors produced by EL08-1D2 cells that support HSC. Thus the more likely explanation for the persistent skewing seen in Wnt5a supplemented EL08-1D2-non-contact, is that aside from Wnt5a, UG26-1B6 cells secrete additional factors that support the most primitive CR-LTR-HSC, such as one or more of those descripbed in Example 2.

That HSC are influenced by Wnts has been shown in numerous publications. Most studies have evaluated the role of the typical canonical Wnt, Wnt3a. The role of Wnt5a in hematopoiesis has been less extensively studies. However, several studies evaluating hematopoietic development in vivo and from ESC in vitro found that Wnt5a (and in one study its putative receptors, Fzd4 and Lrp5), not Wnt3a, is expressed at the time of definitive hematopoietic cell development. Austin, et. al. and Van Den Berg, et. al. demonstrated that Wnt5a increases proliferation of primitive murine and human hematopoietic progenitors, respectively. Two studies have addressed the effect of Wnt5a on HSC. Murdoch, et. al. provided evidence that exposure to Wnt5a during in vitro culture did not significantly affect the proliferative and differentiation capacity of human primitive hematopoietic progenitors, but that administration of Wnt5a containing conditioned medium to mice transplanted with human umbilical cord blood CD34+ CD38−Lin− cells significantly increased the repopulation ability of these cells. These studies would suggest that Wnt5a may affect the most primitive compartment of hematopoietic cells. However, the study did not address whether the effect of Wnt5a was directly on HSC. Nemeth, et. al. demonstrated that culture of highly enriched murine HSC with Wnt5a alone under serum free conditions yields an increase in STR-HSC, possibly by increasing the maintenance of a quiescent state of the ex vivo cultured HSC. They further demonstrate that this appears to occur via activation of the non-canonical signaling pathway as no clear-cut increase in β-catenin was observed following incubation of Lin− cells with Wnt5a, and incubation of Lin− cells with Wnt3a and Wnt5a decreased Wnt3a-mediated increase of β-catenin.

It was demonstrated herein that Wnt5a used in combination with SCF and Tpo does not affect committed HPC or STR-HSC, and only affects the maintenance of LTR-HSC. Also, it was determined that the effect of Wnt5a on KLS cells and CD34-KLS cells appears to be via stabilization and accumulation of β-catenin. An increase in activated β-catenin was detected by Western blot of KLS cells, and by immunostaining of single sorted CD34-KLS cells, which also demonstrated an increase in total β-catenin protein expression following addition of Wnt5a. There is recent evidence for activation of the canonical pathway by Wnt5a following its interaction with the Fzd4 receptor and Lrp5. Herein it is demonstrated that a subpopulation of KLS cells expresses Frzd4, and that the Fzd4+KLS cell population contains nearly all LTR-HSC. By contrast Ror2 transcripts were not detected in KLS cells. The reasons for the differences seen in the studies presented herein and those published by Nemeth, et al. are not totally clear. However, the effect of Wnt5a in combination with SCF and Tpo was tested, whereas Nemeth, et al. studied the effect of Wnt5a without additional cytokines. Whether the combination of the three cytokines alters the mechanism through which Wnt5a signals in cells is currently not known. Moreover, as indicated above Nemeth, et al. evaluated β-catenin activation chiefly in Lin− cells, not more highly enriched KLS or CD34-KLS cells.

In conclusion, it is demonstrated herein that use of stromal feeders from different origin can aid in identifying soluble growth factors/signals that play a role in maintenance of HSC. Comparing the transcriptome of an HSC supportive and a non-supportive feeder identified Wnt5a, as a candidate factor responsible for HSC maintenance. It was demonstrated that Wnt5a can, following addition to non-supportive feeders, or following addition to a feeder free culture system, increase maintenance/expansion of murine HSC in vitro.

### Example 4

### Tissue Factor Pathway Inhibitor: a Novel Molecule for Improving Hematopoietic Stem Cell Engraftment Potential

Stromal cell lines derived from AGM and FL were compared for their potential to maintain hematopoietic stem cells. AGM derived cell line UG26-1B6 was found to be hematopoietic supportive unlike fetal liver derived lines EL08-1D2 and AFT-024. Microarray analysis of differentially expressed genes was done for these cell lines and the screened genes were categorized by their cellular location. 18 secretory genes that showed a high expression in UG26-1B6 were identified. Tissue factor pathway inhibitor (Tfpi) was one of the genes that was differentially expressed.

Sorted BM-KLS cells were cultured for 5 days in the presence of SCF/Tpo with or without Tfpi. As shown by Competitive repopulation assays the cells cultured in the presence of Tfpi showed better chimerism following transplantation. Tfpi acts as an anti-coagulant in vivo (Sandset, Haemostasis 1996; 26:154-165), but its role in hematopoiesis has not been reported. In vitro adhesion experiments were done to check the effect of Tfpi on attachment of KLS cells on BM stromal cells line OP9. Tfpi was found to increase attachment of HSCs on these stromal cells. Tfpi had the same effect on migration of KLS cell towards OP9 cells. Tissue factor (TF) is the receptor for Tfpi in coagulation pathway (Broze, Annual Review of Medicine 1995; 46:103-122). As VEGF is known to increase TF in endothelial cells (Zucker et al. Int J Cancer 1998; 75:780-786), the effect of VEGF induced increased TF expression on KLS cells migration and adhesion was analyzed. The results did not support Tfpi action through TF. Moreover, TF expression on BM derived KLS cells was not detected, which could have led to binding of Tfpi.

Glypican 3 (Gpc3) is a heparan sulphate proteoglycan which is associated with the cytoplasmic membrane through glycosyl phospatidyl inositol (GPI) anchor (Filmus et al. Genome Biology 2008; 9:224). Tfpi is known to bind to Gpc3. It was determined that the expression of Gpc3 is restricted to BM progenitors with higher level of expression on primitive cells. Gpc3 is removed from the cell surface following heparin wash (Berman et al. 1999; 274:36132-36138). Interestingly, when the KLS cells were washed with heparin, Tfpi binding to these cells decreased, suggesting Tfpi could function through Gpc3. Further experiments on CD26 activity supported this hypothesis. Gpc3 inhibits the activity of CD26 (Davoodi et al. Proteomics 2007; 7:2300-2310) which inhibits HSC migration in response to SDF-1α (Christopherson et al. Science 2004; 305:1000-1003). CD26 (dipeptidyl peptidase IV; DPP IV) cleaves and inactivates SDF-1α thereby affecting HSC migration (Christopherson et al. Blood 2003; 101:4680-4686; Lambeir et al. J Biol Chem. 2001; 276:29839-29845). W CD26 activity was measured in sorted BM-KLS cells following culture in the presence of Tfpi. It was observed that addition of Tfpi inhibited CD26 activity in BM-KLS cells. Decrease in CD26 activity has been shown to assist in better engraftment (Campbell et al. Stem Cells and Development 2007; 16:347-354). Regulation of CD26 activity by Tfpi postulates a novel method to increase engraftment potential of BM-HSC.

These studies not only demonstrate a novel mechanism of regulation of HSC engraftmen, but also present the use of Tfpi as a molecule for better engraftment in clinical settings.

### Bibliography

[1] Oostendorp R A, Harvey K N, Kusadasi N, et al. Stromal cell lines from mouse aorta-gonads-mesonephros subregions are potent supporters of hematopoietic stem cell activity. Blood. 2002; 99:1183-1189.
[2] Moore K A, Ema H, Lemischka I R. In vitro maintenance of highly purified, transplantable hematopoietic stem cells. Blood. 1997; 89:4337-4347.
[3] Wineman J, Moore K, Lemischka I, Muller-Sieburg C. Functional heterogeneity of the hematopoietic microenvironment: rare stromal elements maintain long-term repopulating stem cells. Blood. 1996; 87:4082-4090.
[4] Zhao R C, Jiang Y, Verfaillie C M. A model of human p210(bcr/ABL)-mediated chronic myelogenous leukemia by transduction of primary normal human CD34(+) cells with a BCR/ABL-containing retroviral vector. Blood. 2001; 97:2406-2412.
[5] Zhang C C, Lodish H F. Insulin-like growth factor 2 expressed in a novel fetal liver cell population is a growth factor for hematopoietic stem cells. Blood. 2004; 103:2513-2521.
[6] Ema H, Morita Y, Yamazaki S, et al. Adult mouse hematopoietic stem cells: purification and single-cell assays. Nature protocols. 2006; 1:2979-2987.
[7] Baron M H. Embryonic origins of mammalian hematopoiesis. Experimental hematology. 2003; 31:1160-1169.
[8] Palis J, Yoder M C. Yolk-sac hematopoiesis: the first blood cells of mouse and man. Experimental hematology. 2001; 29:927-936.
[9] Dzierzak E, Medvinsky A. Mouse embryonic hematopoiesis. Trends Genet. 1995; 11:359-366.
[10] Calvi L M, Adams G B, Weibrecht K W, et al. Osteoblastic cells regulate the haematopoietic stem cell niche. Nature. 2003; 425:841-846.
[11] Zhang J, Niu C, Ye L, et al. Identification of the haematopoietic stem cell niche and control of the niche size. Nature. 2003; 425:836-841.
[12] Dexter T M, Allen T D, Lajtha L G. Conditions controlling the proliferation of haemopoietic stem cells in vitro. J Cell Physiol. 1977; 91:335-344.
[13] Dexter T M, Coutinho L H, Spooncer E, et al. Stromal cells in haemopoiesis. Ciba Found Symp. 1990; 148:76-86; discussion 86-95.
[14] Dexter T M, Spooncer E, Simmons P, Allen T D. Long-term marrow culture: an overview of techniques and experience. Kroc Found Ser. 1984; 18:57-96.
[15] Gartner S, Kaplan H S. Long-term culture of human bone marrow cells. Proc Natl Acad Sci USA. 1980; 77:4756-4759.
[16] Thiemann F T, Moore K A, Smogorzewska E M, Lemischka I R, Crooks G M. The murine stromal cell line AFT024 acts specifically on human CD34+ CD38− progenitors to maintain primitive function and immunophenotype in vitro. Exp Hematol. 1998; 26:612-619.
[17] Oostendorp R A, Medvinsky A J, Kusadasi N, et al. Embryonal subregion-derived stromal cell lines from novel temperature-sensitive SV40 T antigen transgenic mice support hematopoiesis. J Cell Sci. 2002; 115:2099-2108.
[18] Harvey K, Dzierzak E. Cell-Cell Contact and Anatomical Compatibility in Stromal Cell-Mediated HSC Support During Development. Stem Cells. 2004; 22:253-258.
[19] Kusadasi N, Oostendorp R A, Koevoet W J, Dzierzak E A, Ploemacher R E. Stromal cells from murine embryonic aorta-gonad-mesonephros region, liver and gut mesentery expand human umbilical cord blood-derived CAFC (week6) in extended long-term cultures. Leukemia. 2002; 16:1782-1790.
[20] Harrison D E. Competitive repopulation: a new assay for long-term stem cell functional capacity. Blood. 1980; 55:77-81.
[21] Szilvassy S J, Humphries R K, Lansdorp P M, Eaves A C, Eaves O. Quantitative assay for totipotent reconstituting hematopoietic stem cells by a competitive repopulation strategy. Proc Natl Acad Sci USA. 1990; 87:8736-8740.
[22] Harrison D E, Jordan C T, Zhong R K, Astle C M. Primitive hemopoietic stem cells: direct assay of most productive populations by competitive repopulation with simple binomial, correlation and covariance calculations. Exp Hematol. 1993; 21:206-219.
[23] Muller A M, Medvinsky A, Strouboulis J, Grosveld F, Dzierzak E. Development of hematopoietic stem cell activity in the mouse embryo. Immunity. 1994; 1:291-301.
[24] Dyer M A, Farrington S M, Mohn D, Munday J R, Baron M H. Indian hedgehog activates hematopoiesis and vasculogenesis and can respecify prospective neurectodermal cell fate in the mouse embryo. Development. 2001; 128:1717-1730.
[25] Baron M. Induction of embryonic hematopoietic and endothelial stem/progenitor cells by hedgehog-mediated signals. Differentiation. 2001; 68:175-185.
[26] Bhardwaj G, Murdoch B, Wu D, et al. Sonic hedgehog induces the proliferation of primitive human hematopoietic cells via BMP regulation. Nat. Immunol. 2001; 2:172-180.
[27] Bhatia M, Bonnet D, Wu D, et al. Bone morphogenetic proteins regulate the developmental program of human hematopoietic stem cells. The Journal of experimental medicine. 1999; 189:1139-1148.
[28] Merrill B J, Gat U, DasGupta R, Fuchs E. Tcf3 and Lef1 regulate lineage differentiation of multipotent stem cells in skin. Genes Dev. 2001; 15:1688-1705.
[29] Korinek V, Barker N, Moerer P, et al. Depletion of epithelial stem-cell compartments in the small intestine of mice lacking Tcf-4. Nat. Genet. 1998; 19:379-383.
[30] Lee H Y, Kleber M, Hari L, et al. Instructive role of Wnt/beta-catenin in sensory fate specification in neural crest stem cells. Science. 2004; 303:1020-1023.
[31] Sato N, Meijer L, Skaltsounis L, Greengard P, Brivanlou A H. Maintenance of pluripotency in human and mouse embryonic stem cells through activation of Wnt signaling by a pharmacological GSK-3-specific inhibitor. Nat. Med. 2004; 10:55-63.
[32] Austin T W, Solar G P, Ziegler F C, Liem L, Matthews W. A role for the Wnt gene family in hematopoiesis: expansion of multilineage progenitor cells. Blood. 1997; 89:3624-3635.
[33] Willert K, Brown J D, Danenberg E, et al. Wnt proteins are lipid-modified and can act as stem cell growth factors. Nature. 2003; 423:448-452.
[34] Reya T, Duncan A W, Ailles L, et al. A role for Wnt signalling in self-renewal of haematopoietic stem cells. Nature. 2003; 423:409-414.
[35] Van Den Berg D J, Sharma A K, Bruno E, Hoffman R. Role of members of the Wnt gene family in human hematopoiesis. Blood. 1998; 92:3189-3202.
[36] Orelio C, Dzierzak E. Identification of 2 novel genes developmentally regulated in the mouse aorta-gonad-mesonephros region. Blood. 2003; 101:2246-2249.
[37] Kusadasi N, Koevoet J L, van Soest P L, Ploemacher R E. Stromal support augments extended long-term ex vivo expansion of hemopoietic progenitor cells. Leukemia. 2001; 15:1347-1358.
[38] Oostendorp R A, Robin C, Steinhoff C, et al. Long-term maintenance of hematopoietic stem cells does not require contact with embryo-derived stromal cells in cocultures. Stem cells (Dayton, Ohio). 2005; 23:842-851.
[39] Lewis I D, Almeida-Porada G, Du J, et al. Umbilical cord blood cells capable of engrafting in primary, secondary, and tertiary xenogeneic hosts are preserved after ex vivo culture in a noncontact system. Blood. 2001; 97:3441-3449.
[40] Punzel M, Gupta P, Verfaillie C M. The microenvironment of AFT024 cells maintains primitive human hematopoiesis by counteracting contact mediated inhibition of proliferation. Cell communication & adhesion. 2002; 9:149-159.
[41] Verfaillie C M. Direct contact between human primitive hematopoietic progenitors and bone marrow stroma is not required for long-term in vitro hematopoiesis. Blood. 1992; 79:2821-2826.
[42] Podesta M, Piaggio G, Pitto A, et al. Modified in vitro conditions for cord blood-derived long-term culture-initiating cells. Experimental hematology. 2001; 29:309-314.
[43] Verfaillie C M. Soluble factor(s) produced by human bone marrow stroma increase cytokine-induced proliferation and maturation of primitive hematopoietic progenitors while preventing their terminal differentiation. Blood. 1993; 82:2045-2053.
[44] Yamane T, Kunisada T, Tsukamoto H, et al. Wnt signaling regulates hemopoiesis through stromal cells. J Immunol. 2001; 167:765-772.
[45] Petzer A L, Eaves O, Barnett M J, Eaves A C. Selective expansion of primitive normal hematopoietic cells in cytokine-supplemented cultures of purified cells from patients with chronic myeloid leukemia. Blood. 1997; 90:64-69.
[46] Zandstra P W, Conneally E, Petzer A L, Piret J M, Eaves CI Cytokine manipulation of primitive human hematopoietic cell self-renewal. Proceedings of the National Academy of Sciences of the United States of America. 1997; 94:4698-4703.
[47] Zhang C C, Kaba M, Ge G, et al. Angiopoietin-like proteins stimulate ex vivo expansion of hematopoietic stem cells. Nature medicine. 2006; 12:240-245.
[48] Hackney J A, Charbord P, Brunk B P, Stoeckert O, Lemischka I R, Moore K A. A molecular profile of a hematopoietic stem cell niche. Proceedings of the National Academy of Sciences of the United States of America. 2002; 99:13061-13066.
[49] Huynh H, lizuka S, Kaba M, et al. Insulin-like growth factor-binding protein 2 secreted by a tumorigenic cell line supports ex vivo expansion of mouse hematopoietic stem cells. Stem cells (Dayton, Ohio). 2008; 26:1628-1635.
[50] Zhang C C, Kaba M, lizuka S, Huynh H, Lodish H F. Angiopoietin-like 5 and IGFBP2 stimulate ex vivo expansion of human cord blood hematopoietic stem cells as assayed by NOD/SCID transplantation. Blood. 2008; 111:3415-3423.
[51] Maroney S A, Cooley B C, Sood R, Weiler H, Mast A E. Combined tissue factor pathway inhibitor and thrombomodulin deficiency produces an augmented hypercoagulable state with tissue-specific fibrin deposition. J Thromb Haemost. 2008; 6:111-117.
[52] Maroney S A, Mast A E. Expression of tissue factor pathway inhibitor by endothelial cells and platelets. Transfus Apher Sci. 2008; 38:9-14.
[53] Rydziel S, Stadmeyer L, Zanotti S, Durant D, Smerdel-Ramoya A, Canalis E. Nephroblastoma overexpressed (Nov) inhibits osteoblastogenesis and causes osteopenia. The Journal of biological chemistry. 2007; 282:19762-19772.
[54] Murdoch B, Chadwick K, Martin M, et al. Wnt-5A augments repopulating capacity and primitive hematopoietic development of human blood stem cells in vivo. Proceedings of the National Academy of Sciences of the United States of America. 2003; 100:3422-3427.
[55] Trowbridge J J, Xenocostas A, Moon R T, Bhatia M. Glycogen synthase kinase-3 is an in vivo regulator of hematopoietic stem cell repopulation. Nature medicine. 2006; 12:89-98.
[56] Nemeth M J, Topol L, Anderson S M, Yang Y, Bodine D M. Wnt5a inhibits canonical Wnt signaling in hematopoietic stem cells and enhances repopulation. Proceedings of the National Academy of Sciences of the United States of America. 2007; 104:15436-15441.
[57] Scheller M, Huelsken J, Rosenbauer F, et al. Hematopoietic stem cell and multilineage defects generated by constitutive beta-catenin activation. Nature immunology. 2006; 7:1037-1047.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to maintain/expand hematopoietic stem cells (HSCs) comprising contacting HSCs with at least one or more exogenous factors selected from TFPI, DEFCR3, SERPINE2, COL18A1, BGLAP, COL8A1, INHBA, LTBP2, MAC30, a biologically active fragment or derivative thereof so as to maintain/expand said HSCs.

2. (canceled)

3. The method of claim 1, wherein the one or more factors are SERPINE2 and/or Tfpi.

4. (canceled)

5. The method of claim 1, wherein the HSCs are further contacted with one or more growth factors or cytokines.

6. (canceled)

7. The method of claim 1, wherein the maintained/expanded HSCs are long-term-repopulating (LTR-) HSCs.

8. The method of claim 7, wherein the LTR-HSCs are competitive repopulation (CR)-long-term-repopulating (LTR-) HSC.

9. The method of claim 1, wherein the maintained/expanded HSCs are short-term-repopulating (STR-) HSCs.

10-11. (canceled)

12. Cells produced according to the method of claim 1.

13. A composition comprising the cells of claim 12.

14. A composition comprising HSCs and one or more factors selected from TFPI, DEFCR3, SERPINE2, COL18A1, BGLAP, COL8A1, INHBA, LTBP2, MAC30, a biologically active fragment or derivative thereof.

15. (canceled)

16. The composition of claim 14, where the one or more factors are SERPINE2 and/or Tfpi.

17. The composition of claim 14 further comprising a cytokine or growth factor.

18. (canceled)

19. A method to prepare a composition comprising combining one or more factors selected from TFPI, DEFCR3, SERPINE2, COL18A1, BGLAP, COL8A1, INHBA, LTBP2, MAC30, a biologically active fragment or derivative thereof with HSCs.

20. (canceled)

21. The method of claim 19, wherein the one or more factors are SERPINE2 and/or Tfpi.

22. The method of claim 19 further comprising adding a cytokine or growth factor.

23. (canceled)

24. A method to treat a non-malignant blood disorder, a metabolic storage disorder or cancer comprising administering to a subject in need thereof HSCs which have been contacted with one or more factors selected from TFPI, DEFCR3, SERPINE2, COL18A1, BGLAP, COL8A1, INHBA, LTBP2, MAC30, a biologically active fragment or derivative thereof so as to treat a non-malignant blood disorder, a metabolic storage disorder or cancer in the subject.

25. (canceled)

26. The method of claim 24, where the one or more factors are SERPINE2 and/or Tfpi.

27. The method of claim 24, wherein the HSCs have further been contacted with a cytokine.

28-35. (canceled)

36. A method to treat a non-malignant blood disorder, a metabolic storage disorder or cancer comprising administering to a subject in need thereof one or more factors selected from TFPI, DEFCR3, SERPINE2, COL18A1, BGLAP, COL8A1, INHBA, LTBP2, MAC30, a biologically active fragment or derivative thereof so as to treat a non-malignant blood disorder, a metabolic storage disorder or cancer in the subject.

37. (canceled)

38. The method of claim 36, where the one or more factors is SERPINE2 and/or Tfpi.

39. The method of claim 36, further comprising administering a cytokine.

40-44. (canceled)
